(19) United States
(12) Patent Application Publication
(10) Pub. No.: US 2024/0216334 A1
(43) Pub. Date: Jul. 4, 2024

(54) METHODS AND COMPOSITIONS FOR TREATING MYDRIASIS, GLAUCOMA, AND OTHER OCULARCONDITIONS

(71) Applicant: Ocuphire Pharma, Inc., Farmington Hills, MI (US)

(72) Inventors: Jay Stuart PEPOSE, Chesterfield, MO (US); Eliot Stuart LAZAR, Orchard Park, NY (US); Mina SOOCH, Bloomfield, MI (US); Alan R. MEYER, North Riverdale, IL (US); Konstantinos CHARIZANIS, Ypsilanti, MI (US); Bemhard HOFFMANN, Lake Forest, IL (US); William H. PITLICK, Seattle, WA (US)

(21) Appl. No.: 18/556,599

(22) PCT Filed: Apr. 22, 2022
(86) PCT No.: PCT/US2022/025912

§ 371 Date: Oct. 20, 2023

Related U.S. Application Data
(60) Provisional application No. 63/178,578, filed on Apr. 23, 2021.

(51) Int. Cl. A61K 31/417; A61K 9/00; A61K 9/08; A61K 31/4178; A61K 31/498; A61K 47/10; A61K 47/12; A61K 47/18; A61K 47/26; A61K 47/38; A61P 27/06

(52) U.S. Cl. CPC A61K 31/417; A61K 9/0048; A61K 9/08; A61K 31/4178; A61K 31/498; A61K 47/10; A61K 47/12; A61K 47/186; A61K 47/26; A61K 47/38; A61P 27/06

(57) ABSTRACT

The invention provides methods, compositions, and kits containing an alpha-adrenergic antagonist, such as phentolamine, for treating patients suffering from mydriasis, glaucoma, and other ocular conditions.

### CROSS-REFERENCE TO RELATED APPLICATIONS

This application is the national stage application of International (PCT) Patent Application Serial No. PCT/US2022/025912, filed Apr. 22, 2022, which claims the benefit of and priority to U.S. Provisional Patent Application Ser. No. 63/178,578, filed Apr. 23, 2021; the contents of which are hereby incorporated by reference in their entirety.

### FIELD OF THE INVENTION

The invention provides methods, compositions, and kits containing an alpha-adrenergic antagonist, such as phentolamine, for treating patients suffering from mydriasis, glaucoma, and other ocular conditions.

### BACKGROUND

Mydriasis is a disorder of the eye characterized by an unusually dilated pupil, frequently caused by one or more of disease, trauma, or a pharmacological agent. The pharmacological agent could be, for example, an agent administered to the eye to cause pupil dilation as part of an eye examination. Alternatively, the pharmacological agent could be an agent administered to the patient for other reasons, and may be a single administration of the agent to the patient or an agent administered on multiple occasions. The negative effects of mydriasis can include sensitivity to light and inability to focus, particularly in bright environments. Existing treatments for mydriasis vary, based on the cause of the mydriasis, but are not effective for all patients and/or have undesirable characteristics. Better treatments are needed for mydriasis.

Glaucoma is a disease of the eye that often affects the retina and/or optic nerve and, if left untreated, can lead to blindness. Prolonged periods of elevated intraocular pressure are a common characteristic of many forms of glaucoma. Such prolonged periods of elevated intraocular pressure can result in irreversible damage to the retina and optic nerve, resulting in progressive, permanent vision loss. Treatments that reduce intraocular pressure provide benefits to patients suffering from glaucoma. However, existing drug therapies to reduce intraocular pressure are not effective for all patients and/or have undesirable side effects. Better treatments are needed for glaucoma.

Deficient visual performance under dim light conditions can have a significant negative impact on a patient's quality of life, affecting, for example, ability to operate a motor vehicle at night. Patients that are more likely to experience night vision problems include those suffering from night myopia, have an equatorial cortical cataract, have had surgery to insert an intraocular lens, and/or underwent LASIK surgery. Exemplary symptoms of poor night vision include glare, halos, starburst, ghosting patterns, and/or poor depth perception. Treatments are needed to improve patient visual performance under dim light conditions.

The present invention addresses the aforementioned need for methods and compositions for treating patients suffering mydriasis, glaucoma, and other ocular conditions, and the invention provides other related advantages.

### SUMMARY

The invention provides methods, compositions, and kits containing an alpha-adrenergic antagonist, such as phentolamine, for treating patients suffering from mydriasis, glaucoma, and other ocular conditions. The alpha-adrenergic antagonist, such as phentolamine, is administered topically to the eye of the patient, preferably in the form of a liquid aqueous ophthalmic formulation. In certain embodiments, a muscarinic acetylcholine receptor agonist, such as pilocarpine or a pharmaceutically acceptable salt thereof, is administered topically to the eye of the patient. Exemplary aspects and embodiments of the invention are described below.

One aspect of the invention provides a method of treating mydriasis in a patient. The method comprises administering to an eye of a patient in need thereof a dosage of an alpha-adrenergic antagonist and a dosage of a muscarinic acetylcholine receptor agonist in an amount effective to thereby treat the mydriasis. In certain embodiments, the alpha-adrenergic antagonist is phentolamine mesylate. In certain embodiments, the muscarinic acetylcholine receptor agonist is pilocarpine or a pharmaceutically acceptable salt thereof. In certain embodiments, the muscarinic acetylcholine receptor agonist is pilocarpine hydrochloride.

Another aspect of the invention provides a method of treating mydriasis in a patient suffering from glaucoma while reducing the risk of an angle-closure glaucoma attack. The method comprises administering to an eye of a patient in need thereof a dosage of an alpha-adrenergic antagonist in an amount effective to thereby treat the mydriasis and reduce the risk of an angle-closure glaucoma attack. In certain embodiments, the alpha-adrenergic antagonist is phentolamine mesylate. In certain embodiments, the muscarinic acetylcholine receptor agonist is pilocarpine or a pharmaceutically acceptable salt thereof. In certain embodiments, the muscarinic acetylcholine receptor agonist is pilocarpine hydrochloride.

Another aspect of the invention provides a method of improving visual performance under dim light conditions in a patient. The method comprises administering to an eye of a patient in need thereof a dosage of an alpha-adrenergic antagonist and a dosage of a muscarinic acetylcholine receptor agonist in an amount effective to thereby improve visual performance under dim light conditions. In certain embodiments, the alpha-adrenergic antagonist is phentolamine mesylate. In certain embodiments, the muscarinic acetylcholine receptor agonist is pilocarpine or a pharmaceutically acceptable salt thereof. In certain embodiments, the muscarinic acetylcholine receptor agonist is pilocarpine hydrochloride.

Another aspect of the invention provides a method of reducing pupil diameter under dim light conditions in a patient. The method comprises administering to an eye of a patient in need thereof a dosage of an alpha-adrenergic antagonist and a dosage of a muscarinic acetylcholine receptor agonist in an amount effective to thereby reduce pupil diameter under dim light conditions. In certain embodiments, the alpha-adrenergic antagonist is phentolamine mesylate. In certain embodiments, the muscarinic acetylcholine receptor agonist is pilocarpine or a pharmaceutically acceptable salt thereof. In certain embodiments, the muscarinic acetylcholine receptor agonist is pilocarpine hydrochloride.

Another aspect of the invention provides a method of reducing an aberrant focus of scattered light rays in a patient's eye under dim light conditions. The method comprises administering to an eye of a patient in need thereof a dosage of an alpha-adrenergic antagonist and a dosage of a muscarinic acetylcholine receptor agonist in an amount effective to thereby reduce aberrant focus of scattered light rays in a patient's eye under dim light conditions. In certain embodiments, the alpha-adrenergic antagonist is phentolamine mesylate. In certain embodiments, the muscarinic acetylcholine receptor agonist is pilocarpine or a pharmaceutically acceptable salt thereof. In certain embodiments, the muscarinic acetylcholine receptor agonist is pilocarpine hydrochloride.

Another aspect of the invention provides a method of treating acute angle-closure glaucoma in a patient. The method comprises administering to an eye of a patient in need thereof a dosage of an alpha-adrenergic antagonist selected from phentolamine or a pharmaceutically acceptable salt thereof in an amount effective to treat the acute angle-closure glaucoma. In certain embodiments, the alpha-adrenergic antagonist is phentolamine mesylate. In certain embodiments, the method further comprises administering to the eye of the patient a muscarinic acetylcholine receptor agonist. In certain embodiments, the muscarinic acetylcholine receptor agonist is pilocarpine or a pharmaceutically acceptable salt thereof. In certain embodiments, the muscarinic acetylcholine receptor agonist is pilocarpine hydrochloride.

Another aspect of the invention provides a method of preventing angle-closure glaucoma in a patient. The method comprises administering to an eye of a patient in need thereof a dosage of an alpha-adrenergic antagonist selected from phentolamine or a pharmaceutically acceptable salt thereof in an amount effective to prevent angle-closure glaucoma. In certain embodiments, the alpha-adrenergic antagonist is phentolamine mesylate. In certain embodiments, the method further comprises administering to the eye of the patient a muscarinic acetylcholine receptor agonist. In certain embodiments, the muscarinic acetylcholine receptor agonist is pilocarpine or a pharmaceutically acceptable salt thereof. In certain embodiments, the muscarinic acetylcholine receptor agonist is pilocarpine hydrochloride.

Another aspect of the invention provides a method of treating or preventing a narrow angle attack in a patient. The method comprises administering to an eye of a patient in need thereof a dosage of an alpha-adrenergic antagonist selected from phentolamine or a pharmaceutically acceptable salt thereof in an amount effective to treat or prevent the narrow angle attack. In certain embodiments, the patient's eye has a narrow angle. In certain embodiments, the alpha-adrenergic antagonist is phentolamine mesylate. In certain embodiments, the method further comprises administering to the eye of the patient a muscarinic acetylcholine receptor agonist. In certain embodiments, the muscarinic acetylcholine receptor agonist is pilocarpine or a pharmaceutically acceptable salt thereof. In certain embodiments, the muscarinic acetylcholine receptor agonist is pilocarpine hydrochloride.

### BRIEF DESCRIPTION OF FIGURES

FIG. 1 depicts exemplary eye redness as measured according to (1) the CCLRU Redness Grading Scale, and (2) the NYX-001 Redness Grading Scale.

### DETAILED DESCRIPTION OF THE INVENTION

The invention provides methods, compositions, and kits containing an alpha-adrenergic antagonist, such as phentolamine, for treating patients suffering from mydriasis, glaucoma, and other ocular conditions. The alpha-adrenergic antagonist, such as phentolamine, is administered topically to the eye of the patient, preferably in the form of a liquid aqueous ophthalmic formulation. In certain embodiments, a muscarinic acetylcholine receptor agonist, such as pilocarpine or a pharmaceutically acceptable salt thereof, is administered topically to the eye of the patient. Various aspects of the invention are set forth below in sections; however, aspects of the invention described in one particular section are not to be limited to any particular section.

### Definitions

To facilitate an understanding of the present invention, a number of terms and phrases are defined below.

The terms “a,” “an” and “the” as used herein mean “one or more” and include the plural unless the context is inappropriate.

As used herein, the term “patient” refers to organisms to be treated by the methods of the present invention. Such organisms preferably include, but are not limited to, mammals (e.g., murines, simians, equines, bovines, porcines, canines, felines, and the like), and most preferably includes humans.

As used herein, the term “effective amount” refers to the amount of a compound sufficient to effect beneficial or desired results. Unless specified otherwise, an effective amount can be administered in one or more administrations, applications or dosages and is not intended to be limited to a particular formulation or administration route. As used herein, the term “treating” includes any effect, e.g., lessening, reducing, modulating, ameliorating or eliminating, that results in the improvement of the condition, disease, disorder, and the like, or ameliorating a symptom thereof.

As used herein, the term “pharmaceutical composition” refers to the combination of an active agent with a carrier, inert or active, making the composition especially suitable for therapeutic use in vivo or ex vivo.

As used herein, the term “pharmaceutically acceptable carrier” refers to any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see Martin in Remington's Pharmaceutical Sciences, 15th Ed., Mack Publ. Co., Easton, PA [1975].

As used herein, the term “pharmaceutically acceptable salt” refers to any pharmaceutically acceptable salt (e.g., acid or base) of a compound of the present invention which, upon administration to a subject, is capable of providing a compound of this invention. As is known to those of skill in the art, “salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW₃, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate (mesylate), 2-naphthalenesulfonate, nicotinate, oxalate, palmoate, pectinate, persulfate, phenylpropionate, picrate, pivalate, propionate, succinate, sulfate, tartrate, thiocyanate, tosylate, undecanoate, and the like. Other examples of salts include anions of the compounds of the present invention compounded with a suitable cation such as Na⁺, NH₄⁺, and NW₄⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The term “alkanoate” is art-recognized and refers to alkyl-C(O)O—.

The term “alkyl” is art-recognized, and includes saturated aliphatic groups, including straight-chain alkyl groups, branched-chain alkyl groups, cycloalkyl (alicyclic) groups, alkyl substituted cycloalkyl groups, and cycloalkyl substituted alkyl groups. In certain embodiments, a straight chain or branched chain alkyl has about 30 or fewer carbon atoms in its backbone (e.g., C₁-C₃₀ for straight chain, C₃-C₃₀ for branched chain), and alternatively, about 20 or fewer. Likewise, cycloalkyls have from about 3 to about 10 carbon atoms in their ring structure, and alternatively about 5, 6 or 7 carbons in the ring structure.

The term “about” refers to within ±10% of the stated value. The invention encompasses embodiments where the value is within ±9%, ±8%, ±7%, ±6%, ±5%, ±4%, ±3%, ±2%, or ±10% of the stated value.

Throughout the description, where compositions and kits are described as having, including, or comprising specific components, or where processes and methods are described as having, including, or comprising specific steps, it is contemplated that, additionally, there are compositions and kits of the present invention that consist essentially of, or consist of, the recited components, and that there are processes and methods according to the present invention that consist essentially of, or consist of, the recited processing steps.

As a general matter, compositions specifying a percentage are by weight unless otherwise specified. Further, if a variable is not accompanied by a definition, then the previous definition of the variable controls.

### I. Therapeutic Methods

The invention provides methods for treating patients suffering from mydriasis, glaucoma, and other ocular conditions by administering to the eye of the patient an alpha-adrenergic antagonist, such as phentolamine. The alpha-adrenergic antagonist is administered topically to the eye of the patient, preferably in the form of a liquid aqueous ophthalmic formulation. In certain embodiments, a muscarinic acetylcholine receptor agonist, such as pilocarpine or a pharmaceutically acceptable salt thereof, is administered topically to the eye of the patient. Various aspects and embodiments of the therapeutic methods are described in the sections below. The sections are arranged for convenience and information in one section is not to be limited to that section, but may be applied to methods in other sections.

### A. First Method

One aspect of the invention provides a method of treating mydriasis in a patient. The method comprises administering to an eye of a patient in need thereof a dosage of an alpha-adrenergic antagonist and a dosage of a muscarinic acetylcholine receptor agonist in an amount effective to thereby treat the mydriasis.

In certain embodiments, the patient suffers from glaucoma. In certain embodiments, the patient suffers from glaucoma and has a narrow angle. In the context of glaucoma, the term angle refers to the drainage area (referred to as angle) formed by the cornea and iris through which ocular fluid flows. A narrow angle refers to the situation where the flow of ocular fluid through the angle is partially obstructed.

In certain embodiments, the method reduces the risk of a narrow angle attack in the patient.

In certain embodiments, the method reduces the risk of angle-closure attack.

The method may be further characterized by additional features, such as the dosing regimen and the identity of the alpha-adrenergic antagonist, the identity of the muscarinic acetylcholine receptor agonist, and dosage amount of the foregoing. The invention embraces all permutations and combinations of these features.

### B. Second Method

One aspect of the invention provides a method of treating mydriasis in a patient suffering from glaucoma while reducing the risk of an angle-closure glaucoma attack. The method comprises administering to an eye of a patient in need thereof a dosage of an alpha-adrenergic antagonist in an amount effective to thereby treat the mydriasis and reduce the risk of an angle-closure glaucoma attack.

In certain embodiments, the patient suffers from glaucoma and has a narrow angle.

In certain embodiments, the method further comprises administering to the eye of the patient in need thereof a muscarinic acetylcholine receptor agonist.

The method may be further characterized by additional features, such as the dosing regimen and the identity of the alpha-adrenergic antagonist, the identity of any muscarinic acetylcholine receptor agonist, and dosage amount of the foregoing. The invention embraces all permutations and combinations of these features.

### C. Third Method

One aspect of the invention provides a method of improving visual performance under dim light conditions in a patient. The method comprises administering to an eye of a patient in need thereof a dosage of an alpha-adrenergic antagonist and a dosage of a muscarinic acetylcholine receptor agonist in an amount effective to thereby improve visual performance under dim light conditions.

In certain embodiments, the improvement in visual performance provided by the method is improved visual acuity. In certain embodiments, the method results in an improvement in visual acuity characterized by at least a one-line improvement in the patient's vision measured using a Snellen chart. In certain embodiments, the method results in an improvement in visual acuity characterized by at least a two-line improvement in the patient's vision measured using a Snellen chart.

In certain embodiments, the improvement in visual performance provided by the method is improved contrast sensitivity. Benefits provided by the therapeutic methods can be characterized according to the patient's improvement in contrast sensitivity. For example, in certain embodiments, the improvement in contrast sensitivity is at least a 10% (or 20%, 30%, 50%, 60%, or 70%) improvement measured under mesopic conditions using the Holladay Automated Contrast Sensitivity System. In certain embodiments, the improvement in contrast sensitivity is at least a 10% (or 20%, 30%, 50%, 60%, or 70%) improvement measured under photopic conditions using the Holladay Automated Contrast Sensitivity System. In certain other embodiments, the improvement in contrast sensitivity is at least a 10% (or 20%, 30%, 50%, 60%, or 70%) improvement measured under mesopic conditions or scotopic conditions using the Holladay Automated Contrast Sensitivity System.

In certain other embodiments, the improvement in visual performance provided by the method is both (i) improved visual acuity and (ii) improved contrast sensitivity.

The method may be further characterized by additional features, such as the dosing regimen and the identity of the alpha-adrenergic antagonist, the identity of the muscarinic acetylcholine receptor agonist, and dosage amount of the foregoing. The invention embraces all permutations and combinations of these features.

### D. Fourth Method

One aspect of the invention provides a method of reducing pupil diameter under dim light conditions in a patient. The method comprises administering to an eye of a patient in need thereof a dosage of an alpha-adrenergic antagonist and a dosage of a muscarinic acetylcholine receptor agonist in an amount effective to thereby reduce pupil diameter under dim light conditions.

In certain embodiments, the method results in at least a 20% reduction in pupil diameter under dim light conditions. In certain embodiments, the method results in at least a 30% reduction in pupil diameter under dim light conditions. In certain embodiments, the method results in at least a 35% reduction in pupil diameter under dim light conditions.

The method may be further characterized by additional features, such as the dosing regimen and the identity of the alpha-adrenergic antagonist, the identity of the muscarinic acetylcholine receptor agonist, and dosage amount of the foregoing. The invention embraces all permutations and combinations of these features.

### E. Fifth Method

One aspect of the invention provides a method of reducing an aberrant focus of scattered light rays in a patient's eye under dim light conditions. The method comprises administering to an eye of a patient in need thereof a dosage of an alpha-adrenergic antagonist and a dosage of a muscarinic acetylcholine receptor agonist in an amount effective to thereby reduce aberrant focus of scattered light rays in a patient's eye under dim light conditions.

In certain embodiments, the method reduces aberrant focus of scattered light rays in a patient's eye under dim light conditions for at least 3 hours. In certain embodiments, the method reduces aberrant focus of scattered light rays in a patient's eye under dim light conditions for at least 6 hours. In certain embodiments, the method reduces aberrant focus of scattered light rays under dim light conditions in a patient's eye for at least 12 hours. In certain embodiments, the method reduces aberrant focus of scattered light rays under dim light conditions in a patient's eye for at least 24 hours.

The method may be further characterized by additional features, such as the dosing regimen and the identity of the alpha-adrenergic antagonist, the identity of the muscarinic acetylcholine receptor agonist, and dosage amount of the foregoing. The invention embraces all permutations and combinations of these features.

### F. Sixth Method

One aspect of the invention provides a method of treating acute angle-closure glaucoma in a patient, comprising administering to an eye of a patient in need thereof a dosage of an alpha-adrenergic antagonist selected from phentolamine or a pharmaceutically acceptable salt thereof in an amount effective to treat the acute angle-closure glaucoma.

In certain embodiments, the patient presents with at least two conditions selected from the group consisting of severe eye pain, red eye, reduced vision, and blurred vision.

In certain embodiments, the method further comprises administering to the eye of the patient a muscarinic acetylcholine receptor agonist.

The method may be further characterized by additional features, such as the dosing regimen and the identity of the alpha-adrenergic antagonist, the identity of any muscarinic acetylcholine receptor agonist, and dosage amount of the foregoing. The invention embraces all permutations and combinations of these features.

### G. Seventh Method

One aspect of the invention provides a method of preventing angle-closure glaucoma in a patient. The method comprises administering to an eye of a patient in need thereof a dosage of an alpha-adrenergic antagonist selected from phentolamine or a pharmaceutically acceptable salt thereof in an amount effective to prevent angle-closure glaucoma.

In certain embodiments, the angle-closure glaucoma is acute angle-closure glaucoma.

In certain embodiments, the method further comprises administering to the eye of the patient a muscarinic acetylcholine receptor agonist.

The method may be further characterized according to the dosing regimen. For example, in certain embodiments, the dosage of alpha-adrenergic antagonist is administered for at least three consecutive days. In certain embodiments, the dosage of alpha-adrenergic antagonist is administered for at least seven consecutive days. In certain embodiments, the dosage of alpha-adrenergic antagonist is administered for at least 14 consecutive days. In certain embodiments, the dosage of alpha-adrenergic antagonist is administered on at least three days in a five day period. In certain embodiments, the dosage of alpha-adrenergic antagonist is administered on at least three days in a seven day period. In certain embodiments, the dosage of alpha-adrenergic antagonist is administered on one day in a three day period. In certain embodiments, the dosage of alpha-adrenergic antagonist is administered on one day in a five day period.

The method may be further characterized by additional features, such as the dosing regimen and the identity of the alpha-adrenergic antagonist, the identity of any muscarinic acetylcholine receptor agonist, and dosage amount of the foregoing. The invention embraces all permutations and combinations of these features.

### H. Eighth Method

One aspect of the invention provides a method of treating or preventing a narrow angle attack in a patient. The method comprises administering to an eye of a patient in need thereof a dosage of an alpha-adrenergic antagonist selected from phentolamine or a pharmaceutically acceptable salt thereof in an amount effective to treat or prevent the narrow angle attack.

In certain embodiments, the method further comprises administering to the eye of the patient a muscarinic acetylcholine receptor agonist.

The method may be further characterized according to the dosing regimen. For example, in certain embodiments, the dosage of alpha-adrenergic antagonist is administered for at least three consecutive days. In certain embodiments, the dosage of alpha-adrenergic antagonist is administered for at least seven consecutive days. In certain embodiments, the dosage of alpha-adrenergic antagonist is administered for at least 14 consecutive days. In certain embodiments, the dosage of alpha-adrenergic antagonist is administered on at least three days in a five day period. In certain embodiments, the dosage of alpha-adrenergic antagonist is administered on at least three days in a seven day period. In certain embodiments, the dosage of alpha-adrenergic antagonist is administered on one day in a three day period. In certain embodiments, the dosage of alpha-adrenergic antagonist is administered on one day in a five day period.

The method may be further characterized by additional features, such as the dosing regimen and the identity of the alpha-adrenergic antagonist, the identity of any muscarinic acetylcholine receptor agonist, and dosage amount of the foregoing. The invention embraces all permutations and combinations of these features.

### I. Additional Features of First and Second Therapeutic Methods

The First and Second Therapeutic Methods may be further characterized according to, for example, the type of mydriasis. In certain embodiments, the mydriasis is pharmacologically induced mydriasis. In certain embodiments, the mydriasis is due to the patient having received one or more of atropine, cyclopentolate, homatropine, scopolamine, tropicamide, flubiprofen, suprofen, hydroxyamphetamine, phenylephrine, ketorolac, or a pharmaceutically acceptable salt thereof. In certain embodiments, the mydriasis is due to the patient having received one or more of atropine, cyclopentolate, homatropine, scopolamine, tropicamide, or a pharmaceutically acceptable salt thereof. In certain embodiments, the mydriasis is due to the patient having received one or more of tropicamide, phenylephrine, or a pharmaceutically acceptable salt thereof.

### J. Additional Features of First, Second, Sixth, Seventh, and Eighth Therapeutic Methods

The First, Second, Sixth, Seventh, and Eighth Therapeutic Methods may be further characterized according to, for example, improvements in the patient's visual performance, reduction in pupil diameter, and/or reduction in aberrant focus of scattered light rays in a patient's eye.

### Improvement in Visual Performance

One benefit of the therapeutic methods is that the patient may also experience an improvement in visual performance. Visual performance pertains to the patient's overall vision quality and includes a patient's ability to see clearly, as well as ability to distinguish between an object and its background.

One aspect of visual performance is visual acuity. Visual acuity is a measure of a patient's ability to see clearly. Visual acuity can be measured using, for example, a Snellen chart. Further, the visual acuity measurement can be taken under scotopic conditions, mesopic conditions, and/or photopic conditions.

Another aspect of visual performance is contrast sensitivity. Contrast sensitivity is a measure of the patient's ability to distinguish between an object and its background. Contrast sensitivity can be measured using, for example, a Holladay Automated Contrast Sensitivity System. The contrast sensitivity can be measured under various light conditions, including, for example, photopic conditions, mesopic conditions, and scotopic conditions, each either with or without glare. In certain embodiments, the contrast sensitivity is measured under mesopic conditions either with or without glare.

In certain embodiments, the improvement in visual performance provided by the method is improved visual acuity. In certain embodiments, the improvement in visual performance provided by the method is improved visual acuity under scotopic conditions. In certain embodiments, the improvement in visual performance provided by the method is improved visual acuity under mesopic conditions. In certain embodiments, the improvement in visual performance provided by the method is improved visual acuity under photopic conditions. In certain embodiments, the improvement in visual acuity is a two-line improvement in the patient's vision as measured using the Snellen chart. In certain other embodiments, the improvement in visual acuity is a one-line improvement in the patient's vision as measured using the Snellen chart.

In certain embodiments, the improvement in visual performance provided by the method is improved contrast sensitivity. The improvement in contrast sensitivity can be measured under various light conditions, such as photopic conditions, mesopic conditions, and scotopic conditions. In certain embodiments, the improvement in visual performance provided by the method is improved contrast sensitivity under photopic conditions. In certain embodiments, the improvement in visual performance provided by the method is improved contrast sensitivity under mesopic conditions. In certain embodiments, the improvement in visual performance provided by the method is improved contrast sensitivity under scotopic conditions. Further, contrast sensitivity can be measured in the presence of glare or the absence of glare. All combinations of light conditions and glare are contemplated.

Benefits provided by the therapeutic methods can be characterized according to the patient's improvement in contrast sensitivity. For example, in certain embodiments, the improvement in contrast sensitivity is at least a 10% (or 20%, 30%, 50%, 60%, or 70%) improvement measured under mesopic conditions using the Holladay Automated Contrast Sensitivity System. In certain embodiments, the improvement in contrast sensitivity is at least a 10% (or 20%, 30%, 50%, 60%, or 70%) improvement measured under photopic conditions using the Holladay Automated Contrast Sensitivity System. In certain other embodiments, the improvement in contrast sensitivity is at least a 10% (or 20%, 30%, 50%, 60%, or 70%) improvement measured under mesopic conditions or scotopic conditions using the Holladay Automated Contrast Sensitivity System.

In certain other embodiments, the improvement in visual performance provided by the method is both (i) improved visual acuity (such as under scotopic conditions, mesopic conditions, and/or photopic conditions) and (ii) improved contrast sensitivity (such as under scotopic conditions, mesopic conditions, and/or photopic conditions).

In certain embodiments, the improvement in visual performance is improvement in near-vision performance. In certain embodiments, the improvement in visual performance is improvement in visual performance at a distance. In certain embodiments, the improvement in visual performance is improved visual performance under low-light conditions. In certain embodiments, the improvement in visual performance is improved visual acuity. In certain embodiments, the improvement in visual performance is improved contrast sensitivity. In certain embodiments, the method provides at least a 10% reduction in pupil diameter in the eye of the patient. In certain embodiments, the method provides at least a 15% reduction in pupil diameter in the eye of the patient. In certain embodiments, the method provides at least a 20% reduction in pupil diameter in the eye of the patient. In certain embodiments, the method provides at least a 25% reduction in pupil diameter in the eye of the patient. In certain embodiments, the method provides at least a 30% reduction in pupil diameter in the eye of the patient.

### Reduction in Pupil Diameter

One benefit of the therapeutic methods is that the patient may also experience a reduction in pupil diameter. Reduction in pupil diameter can result in improvement in visual performance.

The reduction in pupil diameter can be characterized according to, for example, the percent reduction in pupil diameter and size of the pupil measured under certain light conditions. Accordingly, in certain embodiments, the reduction in pupil diameter under mesopic conditions is at least 5% compared to the pupil diameter of the patient under the same mesopic conditions but not having received the therapy defined by the method. In certain other embodiments, the reduction in pupil diameter under mesopic conditions is at least 10% compared to the pupil diameter of the patient under the same mesopic conditions but not having received the therapy defined by the method. In certain other embodiments, the patient experiences a reduction in pupil diameter of at least 0.5 mm when measured under mesopic conditions relative to the diameter of the patient's pupil under the same mesopic conditions but not having received the therapy defined by the method. In certain other embodiments, the patient experiences a reduction in pupil diameter ranging from about 0.6 mm to about 3 mm, about 0.6 mm to about 2.5 mm, or about 0.6 mm to about 2 mm when measured under mesopic conditions relative to the diameter of the patient's pupil under the same mesopic conditions but not having received the therapy defined by the method. In certain other embodiments, the patient experiences a reduction in pupil diameter ranging from about 0.6 mm to about 1.2 mm when measured under mesopic conditions relative to the diameter of the patient's pupil under the same mesopic conditions but not having received the therapy defined by the method. In yet other embodiments, the patient's pupil is reduced to a diameter of about 3 mm to about 5 mm, about 3 mm to about 6 mm, about 4 mm to about 5 mm, about 4 mm to about 6 mm, or about 4 mm to about 7 mm under mesopic conditions due to the therapy defined by the method. In certain embodiments, the patient's pupil is reduced to a diameter of about 4 mm to about 6 mm under mesopic conditions due to the therapy defined by the method.

In certain embodiments, the method provides at least a 10% reduction in pupil diameter in the eye of the patient. In certain embodiments, the method provides at least a 15% reduction in pupil diameter in the eye of the patient. In certain embodiments, the method provides at least a 20% reduction in pupil diameter in the eye of the patient. In certain embodiments, the method provides at least a 25% reduction in pupil diameter in the eye of the patient. In certain embodiments, the method provides at least a 30% reduction in pupil diameter in the eye of the patient.

In certain embodiments, the method achieves a pupil diameter in the range of from about 2.5 mm to about 5.5 mm under dim light conditions. In certain embodiments, the method achieves a pupil diameter in the range of from about 3 mm to about 5 mm under dim light conditions. In certain embodiments, the method achieves a pupil diameter in the range of from about 3 mm to about 4.5 mm under dim light conditions.

### Reducing Aberrant Focus of Scattered Light Rays in a Patient's Eye

One benefit of the therapeutic methods is that the patient may also experience a reduction in aberrant focus of scattered light rays in the patient's eye. This can provide improvement in visual performance for the patient. In certain embodiments, the therapeutic method provides a reduction in aberrant focus of scattered light rays in a patient's eye for at least twenty hours. In certain embodiments, the therapeutic method provides a reduction aberrant focus of scattered light rays in a patient's eye for at least twenty-four hours. In yet other embodiments, the therapeutic method provides a reduction aberrant focus of scattered light rays in a patient's eye for at least thirty-six hours, forty-eight hours, sixty hours, or seventy-two hours.

### K. General Considerations for Therapeutic Methods

General considerations that may be applied to therapeutic methods described herein (e.g., the methods described in Parts A-J above) are provided below and include, for example, identity of the alpha-adrenergic antagonist, frequency of administration of the alpha-adrenergic antagonist, the dosage of the alpha-adrenergic antagonist, the identity of the muscarinic acetylcholine receptor agonist, the degree of eye redness, the reduction in intraocular pressure in the eye due to the method, the duration of reduction in intraocular pressure, and patient populations that may derive particular benefits from the therapeutic methods. A more thorough description of such features is provided below. The invention embraces all permutations and combinations of these features.

### Identity of the Alpha-Adrenergic Antagonist

Methods may be further characterized according to the identity of the alpha-adrenergic antagonist. For example, in certain embodiments, the alpha-adrenergic antagonist is phentolamine, phenoxybenzamine, tolazoline, trazodone, alfuzosin, doxazosin, prazosin, tamsulosin, terazosin, silodosin, atipamezole, idazoxan, mirtazapine, yohimbine, fenoldopam, thymoxamine, or a pharmaceutically acceptable salt of any of the foregoing. In certain embodiments, the alpha-adrenergic antagonist is phentolamine or a pharmaceutically acceptable salt thereof. In certain embodiments, the alpha-adrenergic antagonist is a pharmaceutically acceptable salt of phentolamine. In certain embodiments, the alpha-adrenergic antagonist is phentolamine mesylate. In certain embodiments, the alpha-adrenergic antagonist is fenoldopam mesylate.

In certain embodiments, the alpha-adrenergic antagonist is a non-selective alpha-adrenergic antagonist. In certain embodiments, the alpha-adrenergic antagonist is a reversible, non-selective alpha-adrenergic antagonist.

In certain embodiments, the alpha-adrenergic antagonist is characterized according to its activity towards certain alpha-adrenergic receptors. Accordingly, in certain embodiments, the alpha-adrenergic antagonist has antagonist activity towards an alpha-1 adrenergic receptor. Activity toward the alpha-1 adrenergic receptor may be further characterized according to whether there is activity toward one or more of the alpha-1 adrenergic receptor subtypes (e.g., alpha-TA, alpha-1B, and alpha-1D). Accordingly, in certain embodiments, the alpha-adrenergic antagonist has antagonist activity towards the alpha-TA adrenergic receptor. In certain embodiments, the alpha-adrenergic antagonist has antagonist activity towards the alpha-1B adrenergic receptor. In certain embodiments, the alpha-adrenergic antagonist has antagonist activity towards the alpha-1D adrenergic receptor. In certain embodiments, the alpha-adrenergic antagonist has antagonist activity towards each of the alpha-1 adrenergic receptor subtypes.

In certain embodiments, the alpha-adrenergic antagonist has antagonist activity towards an alpha-2 adrenergic receptor. Activity toward the alpha-2 adrenergic receptor may be further characterized according to whether there is activity toward one or more of the alpha-2 adrenergic receptor subtypes (e.g., alpha-2A, alpha-2B, and alpha-2C). Accordingly, in certain embodiments, the alpha-adrenergic antagonist has antagonist activity towards the alpha-2A adrenergic receptor. In certain embodiments, the alpha-adrenergic antagonist has antagonist activity towards the alpha-2B adrenergic receptor. In certain embodiments, the alpha-adrenergic antagonist has antagonist activity towards the alpha-2C adrenergic receptor. In certain embodiments, the alpha-adrenergic antagonist has antagonist activity towards each of the alpha-2 adrenergic receptor subtypes.

The alpha-adrenergic antagonist may be characterized according to its activity towards (i) an alpha-1 adrenergic receptor versus (ii) an alpha-2 adrenergic receptor. In certain embodiments, the alpha-adrenergic antagonist has antagonist activity at both (i) an alpha-1 adrenergic receptor and (ii) an alpha-2 adrenergic receptor. In certain embodiments, the alpha-adrenergic antagonist has antagonist activity at (i) an alpha-1 adrenergic receptor but not (ii) an alpha-2 adrenergic receptor. In certain embodiments, the alpha-adrenergic antagonist has antagonist activity at (i) an alpha-2 adrenergic receptor but not (ii) an alpha-1 adrenergic receptor. In certain embodiments, the inhibitory activity (as, for example, measured by an IC₅₀ value) of the alpha-adrenergic antagonist is at least 10-fold greater towards (i) the alpha-1 adrenergic receptor compared to the (ii) alpha-2 adrenergic receptor. In certain embodiments, the inhibitory activity (as, for example, measured by an IC₅₀ value) of the alpha-adrenergic antagonist is at least 10-fold greater towards (i) the alpha-2 adrenergic receptor compared to (ii) the alpha-1 adrenergic receptor.

### Frequency of Administration of Alpha-Adrenergic Antagonist

Methods may be further characterized according to the frequency of administration of the alpha-adrenergic antagonist. For example, in certain embodiments, the dosage of alpha-adrenergic antagonist is administered to the eye no more than once per day.

### Dosage of Alpha-Adrenergic Antagonist

Methods may be further characterized according to the dosage of alpha-adrenergic antagonist (e.g., phentolamine or pharmaceutically acceptable salt thereof). For example, in certain embodiments, the dosage of alpha-adrenergic antagonist contains from about 0.1 mg to about 2.0 mg of phentolamine or a pharmaceutically acceptable salt thereof. In certain embodiments, the dosage of alpha-adrenergic antagonist contains from about 0.5 mg to about 1.0 mg of phentolamine or a pharmaceutically acceptable salt thereof. In certain other embodiments, the dosage of alpha-adrenergic antagonist contains from about 0.1 mg to about 2.0 mg of phentolamine mesylate. In certain embodiments, the dosage of alpha-adrenergic antagonist contains from about 0.3 mg to about 0.7 mg of phentolamine mesylate. In certain embodiments, the dosage of alpha-adrenergic antagonist contains about 0.5 mg of phentolamine mesylate. In certain other embodiments, the dosage of alpha-adrenergic antagonist contains from about 0.8 mg to about 1.2 mg of phentolamine mesylate. In certain embodiments, the dosage of alpha-adrenergic antagonist contains about 1 mg of phentolamine mesylate.

In certain embodiments, the dosage of alpha-adrenergic antagonist contains from about 0.3 mg to about 0.6 mg of phentolamine mesylate. In certain embodiments, the dosage of alpha-adrenergic antagonist contains from about 0.3 mg to about 0.4 mg of phentolamine mesylate. In certain embodiments, the dosage of alpha-adrenergic antagonist contains about 0.3 mg of phentolamine mesylate. In certain embodiments, the dosage of alpha-adrenergic antagonist contains from about 0.5 mg to about 0.7 mg of phentolamine mesylate. In certain embodiments, the dosage of alpha-adrenergic antagonist contains from about 0.6 mg to about 0.7 mg of phentolamine mesylate. In certain embodiments, the dosage of alpha-adrenergic antagonist contains about 0.6 mg of phentolamine mesylate. In certain embodiments, the dosage of alpha-adrenergic antagonist is in the form of an ophthalmic solution containing an aqueous pharmaceutically acceptable carrier and phentolamine or a pharmaceutically acceptable salt thereof. In certain embodiments, the dosage of alpha-adrenergic antagonist is in the form of an ophthalmic solution containing an aqueous pharmaceutically acceptable carrier and phentolamine mesylate. In certain embodiments, the dosage of alpha-adrenergic antagonist is in the form of an ophthalmic solution containing an aqueous pharmaceutically acceptable carrier and from about 0.1% (w/v) to about 2% (w/v) phentolamine mesylate. In certain embodiments, the dosage of alpha-adrenergic antagonist is in the form of an ophthalmic solution containing water, mannitol, and phentolamine mesylate. In certain embodiments, the dosage of alpha-adrenergic antagonist is in the form of an ophthalmic solution containing water, mannitol, sodium acetate, and phentolamine mesylate.

In certain embodiments, the dosage of alpha-adrenergic antagonist is one eye drop of a solution containing 1% w/w phentolamine mesylate. In certain embodiments, the dosage of alpha-adrenergic antagonist is two eye drops of a solution containing 1% w/w phentolamine mesylate.

The dosage of alpha-adrenergic antagonist (e.g., phentolamine or a pharmaceutically acceptable salt thereof) is desirably administered to the eye of the patient in the form of an ophthalmic solution, which is delivered to the eye in the form of eye drop. A standard eye drop typically contains from about 0.03 mL to about 0.05 mL of solution.

In certain embodiments, the dosage of alpha-adrenergic antagonist may be in the form of an aqueous ophthalmic solution. For example, in certain embodiments, the dosage of alpha-adrenergic antagonist is an aqueous ophthalmic solution free of a chelating agent containing:
(a) about 0.1% (w/v) to about 2% (w/v) of phentolamine mesylate;
(b) about 1% (w/v) to about 6% (w/v) of at least one polyol compound selected from the group consisting of mannitol, glycerol, and propylene glycol;
(c) about 1 mM to about 6 mM of an alkali metal acetate; and
(d) water;
   wherein the solution has a pH in the range of 4 to 6 and does not contain a chelating agent.

In certain embodiments, the dosage of alpha-adrenergic antagonist is an aqueous ophthalmic solution free of a chelating agent containing:
(a) about 0.5% (w/v) to about 2% (w/v) of phentolamine mesylate;
(b) about 1% (w/v) to about 6% (w/v) of at least one polyol compound selected from the group consisting of mannitol, glycerol, and propylene glycol;
(c) about 1 mM to about 6 mM of an alkali metal acetate; and
(d) water;
   wherein the solution has a pH in the range of 4.5 to 5.5 and does not contain a chelating agent.

In certain embodiments, the at least one polyol is mannitol. In certain embodiments, the solution contains 4% (w/v) mannitol. In certain embodiments, the alkali metal acetate is sodium acetate. In certain embodiments, the solution comprises 3 mM sodium acetate.

In certain embodiments, the dosage of alpha-adrenergic antagonist is an aqueous ophthalmic solution free of a chelating agent containing:
(a) about 0.25% (w/v) to about 2% (w/v) of phentolamine mesylate;
(b) about 3% (w/v) to about 5% (w/v) of mannitol;
(c) about 2 mM to about 4 mM of sodium acetate; and
(d) water;
   wherein the solution has a pH in the range of 4.5 to 5.2 and does not contain a chelating agent.

In certain embodiments, the dosage of alpha-adrenergic antagonist is an aqueous ophthalmic solution free of a chelating agent containing:
(a) about 0.5% (w/v) to about 2% (w/v) of phentolamine mesylate;
(b) about 3% (w/v) to about 5% (w/v) of mannitol;
(c) about 2 mM to about 4 mM of sodium acetate; and
(d) water;
   wherein the solution has a pH in the range of 4.6 to 5.2 and does not contain a chelating agent.

In certain embodiments, the dosage of alpha-adrenergic antagonist is an aqueous ophthalmic solution free of a chelating agent containing:
(a) about 0.5% (w/v) to about 1% (w/v) of phentolamine mesylate;
(b) about 4% mannitol;
(c) about 3 mM sodium acetate; and
(d) water;
   wherein the solution has a pH in the range of 4.6 to 5.2 and does not contain a chelating agent.

In certain embodiments, the dosage of alpha-adrenergic antagonist is an aqueous ophthalmic solution containing:
(a) about 0.25% (w/v) to about 2% (w/v) of phentolamine mesylate;
(b) about 3% (w/v) to about 5% (w/v) of mannitol;
(c) about 1 mM to about 6 mM of sodium acetate; and
(d) water;
   wherein the solution has a pH in the range of 4.5 to 5.2 and does not contain any additional component that is a chelating agent.

In certain embodiments, the dosage of alpha-adrenergic antagonist is an aqueous ophthalmic solution containing:
(a) about 1% (w/v) of phentolamine mesylate;
(b) about 3% (w/v) to about 5% (w/v) of mannitol;
(c) about 2 mM to about 4 mM of a buffer comprising sodium acetate; and
(d) water;
   wherein the solution has a pH in the range of 4.5 to 5.2 and does not contain any additional component that is a chelating agent.

In certain embodiments, the dosage of alpha-adrenergic antagonist is an aqueous ophthalmic solution containing:
(a) about 0.25% (w/v) to about 2% (w/v) of phentolamine mesylate;
(b) about 3% (w/v) to about 5% (w/v) of mannitol;
(c) about 2 mM to about 4 mM of sodium acetate; and
(d) water;
   wherein the solution has a pH in the range of 4.5 to 5.2 and does not contain any additional component that is a chelating agent.

In certain embodiments, the dosage of alpha-adrenergic antagonist is an aqueous ophthalmic solution containing:
(a) about 0.5% (w/v) to about 2% (w/v) of phentolamine mesylate;
(b) about 3% (w/v) to about 5% (w/v) of mannitol;
(c) about 2 mM to about 4 mM of a buffer comprising sodium acetate; and
(d) water;
   wherein the solution has a pH in the range of 4.6 to 5.2 and does not contain any additional component that is a chelating agent.

In certain embodiments, the dosage of alpha-adrenergic antagonist is an aqueous ophthalmic solution containing:
(a) about 0.5% (w/v) to about 1% (w/v) of phentolamine mesylate;
(b) about 4% mannitol;
(c) about 3 mM of a buffer comprising sodium acetate; and
(d) water;
   wherein the solution has a pH in the range of 4.6 to 5.2 and does not contain any additional component that is a chelating agent.

In certain embodiments, the dosage of alpha-adrenergic antagonist is an aqueous ophthalmic solution comprising: (a) about 1% (w/v) of phentolamine mesylate; (b) about 4% (w/v) mannitol; (c) about 3 mM of a buffer comprising sodium acetate; and (d) water; wherein the solution has a pH in the range of 4.5 to 5.5 and does not contain any additional component that is a chelating agent.

In certain embodiments, the dosage of alpha-adrenergic antagonist is an aqueous ophthalmic solution free of a chelating agent comprising: (a) about 1% (w/v) of phentolamine mesylate; (b) about 4% (w/v) mannitol; (c) about 3 mM of a buffer comprising sodium acetate; and (d) water; wherein the solution has a pH in the range of 4.0 to 7.5 and does not contain a chelating agent.

### Identity and Dosage of the Muscarinic Acetylcholine Receptor Agonist

Methods may be further characterized according to the identity and dosage of the muscarinic acetylcholine receptor agonist. For example, in certain embodiments, the muscarinic acetylcholine receptor agonist is pilocarpine or a pharmaceutically acceptable salt thereof. In certain embodiments, the muscarinic acetylcholine receptor agonist is a pharmaceutically acceptable salt of pilocarpine. In certain embodiments, the muscarinic acetylcholine receptor agonist is pilocarpine hydrochloride.

In certain embodiments, the dosage of muscarinic acetylcholine receptor agonist is less than about 2 mg. In certain embodiments, the dosage of muscarinic acetylcholine receptor agonist is less than about 1 mg. In certain embodiments, the dosage of muscarinic acetylcholine receptor agonist is less than about 0.5 mg. In certain embodiments, the dosage of muscarinic acetylcholine receptor agonist is less than about 0.25 mg. In certain embodiments, the dosage of muscarinic acetylcholine receptor agonist is from about 0.1 mg to about 1.5 mg. In certain embodiments, the dosage of muscarinic acetylcholine receptor agonist is from about 0.1 mg to about 1.0 mg. In certain embodiments, the dosage of muscarinic acetylcholine receptor agonist is from about 0.1 mg to about 0.5 mg. In certain embodiments, the dosage of muscarinic acetylcholine receptor agonist is from about 0.1 mg to about 0.3 mg. In certain embodiments, the dosage of muscarinic acetylcholine receptor agonist is from about 0.2 mg to about 0.4 mg. In certain embodiments, the dosage of muscarinic acetylcholine receptor agonist is about 0.2 mg, about 0.25 mg, about 0.3 mg, about 0.35 mg, or about 0.4 mg. In certain embodiments, the dosage of muscarinic acetylcholine receptor agonist is about 0.1 mg, about 0.15 mg, about 0.2 mg, about 0.25 mg, about 0.3 mg, about 0.35 mg, or about 0.4 mg. In certain embodiments, the dosage of muscarinic acetylcholine receptor agonist is from about 0.1 mg to about 0.2 mg. In certain embodiments, the dosage of muscarinic acetylcholine receptor agonist is from about 0.1 mg to about 0.15 mg. In certain embodiments, the dosage of muscarinic acetylcholine receptor agonist is from about 0.05 mg to about 0.15 mg. In certain embodiments, the dosage of muscarinic acetylcholine receptor agonist is from about 0.05 mg to about 0.3 mg. In certain embodiments, the dosage of muscarinic acetylcholine receptor agonist is from about 0.3 mg to about 0.7 mg. In certain embodiments, the dosage of muscarinic acetylcholine receptor agonist is from about 0.3 mg to about 0.8 mg. In certain embodiments, the dosage of muscarinic acetylcholine receptor agonist is from about 0.2 mg to about 0.9 mg. In certain embodiments, the dosage of muscarinic acetylcholine receptor agonist is about 0.5 mg, about 0.6 mg, about 0.7 mg, about 0.8 mg, or about 0.9 mg.

In certain embodiments, the dosage of muscarinic acetylcholine receptor agonist is one eye drop of a solution containing 0.4% w/w pilocarpine hydrochloride. In certain embodiments, the dosage of muscarinic acetylcholine receptor agonist is two eye drops of a solution containing 0.4% w/w pilocarpine hydrochloride. In certain embodiments, the dosage of muscarinic acetylcholine receptor agonist is one eye drop of a solution containing 1.25% w/w pilocarpine hydrochloride.

In certain embodiments, the dosage of muscarinic acetylcholine receptor agonist is administered in the form of an ophthalmic solution containing muscarinic acetylcholine receptor agonist, a lipid, and a pharmaceutically acceptable carrier. In certain embodiments, the muscarinic acetylcholine receptor agonist is present in the ophthalmic solution at a concentration ranging from about 0.1% (w/v) to about 4% (w/v). In certain embodiments, the muscarinic acetylcholine receptor agonist is present in the ophthalmic solution at a concentration ranging from about 0.1% (w/v) to about 2% (w/v). In certain embodiments, the muscarinic acetylcholine receptor agonist is present in the ophthalmic solution at a concentration ranging from about 0.1% (w/v) to about 1% (w/v). In certain embodiments, the muscarinic acetylcholine receptor agonist is present in the ophthalmic solution at a concentration ranging from about 0.2% (w/v) to about 1% (w/v). In certain embodiments, the muscarinic acetylcholine receptor agonist is present in the ophthalmic solution at a concentration ranging from about 0.1% (w/v) to about 0.5% (w/v). In certain embodiments, the muscarinic acetylcholine receptor agonist is present in the ophthalmic solution at a concentration of about 0.4% (w/v). In certain embodiments, the muscarinic acetylcholine receptor agonist is present in the ophthalmic solution at a concentration ranging from about 1% (w/v) to about 1.5% (w/v). In certain embodiments, the muscarinic acetylcholine receptor agonist is present in the ophthalmic solution at a concentration of about 1.25% (w/v).

In certain embodiments, the dosage of muscarinic acetylcholine receptor agonist is topically administered to the eye in the form of an eye drop.

In certain embodiments, the dosage of muscarinic acetylcholine receptor agonist is administered to the eye no more than once per day.

### Administration of a Second Dosage of Muscarinic Acetylcholine Receptor Agonist

Methods may be further characterized according to optionally administering a second dosage of muscarinic acetylcholine receptor agonist to the patient. For example, in certain embodiments, the method further comprises topically administering to the eye of the patient a second dosage of a muscarinic acetylcholine receptor agonist in an amount effective to further reduce diameter of the patient's pupil. In certain embodiments, the second dosage of a muscarinic acetylcholine receptor agonist is an ophthalmic solution comprising muscarinic acetylcholine receptor agonist at a concentration of from about 0.1% (w/v) to about 1% (w/v). In certain embodiments, the second dosage of a muscarinic acetylcholine receptor agonist is an ophthalmic solution comprising muscarinic acetylcholine receptor agonist at a concentration of from about 0.1% (w/v) to about 2% (w/v).

### Reduction in Pupil Diameter

Methods may be further characterized according to the extent of reduction in pupil diameter. For example, in certain embodiments, the patient experiences at least a 1 mm reduction in pupil diameter when measured under photopic conditions due to the method. In certain embodiments, the patient experiences at least a 2 mm reduction in pupil diameter when measured under photopic conditions due to the method. In certain embodiments, the patient experiences at least a 3 mm reduction in pupil diameter when measured under photopic conditions due to the method. In certain embodiments, the patient experiences a reduction in pupil diameter ranging from about 0.5 mm to about 5 mm when measured under photopic conditions due to the method. In certain embodiments, the patient experiences at least a 1 mm reduction in pupil diameter when measured under mesopic conditions due to the method. In certain embodiments, the patient experiences at least a 2 mm reduction in pupil diameter when measured under mesopic conditions due to the method. In certain embodiments, the patient experiences at least a 3 mm reduction in pupil diameter when measured under mesopic conditions due to the method. In certain embodiments, the patient experiences a reduction in pupil diameter ranging from about 0.5 mm to about 5 mm when measured under mesopic conditions due to the method.

In yet other embodiments, the patient's pupil is reduced to a diameter of about 3 mm to about 5 mm, about 3 mm to about 6 mm, about 4 mm to about 5 mm, about 4 mm to about 6 mm, or about 4 mm to about 7 mm under dim light conditions due to the therapy defined by the method. In certain embodiments, the patient's pupil is reduced to a diameter of about 4 mm to about 6 mm under dim light conditions due to the therapy defined by the method.

### Degree of Eye Redness

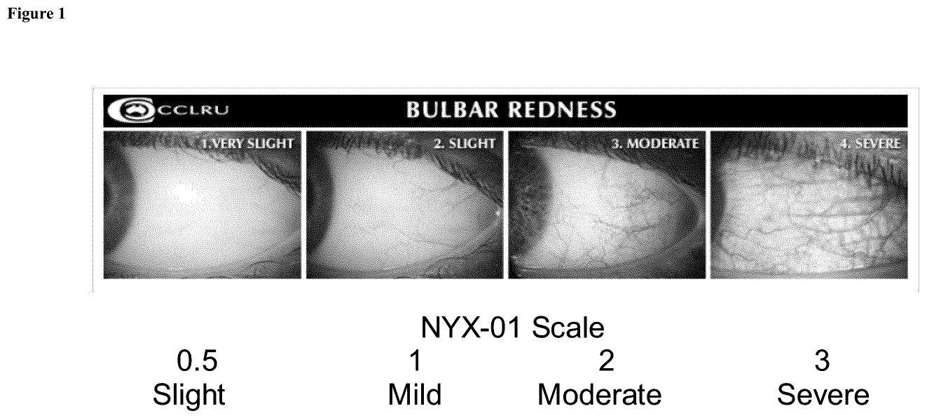

Methods may be further characterized according to the degree of eye redness the patient experiences. The degree of eye redness can be evaluated and characterized using procedures described in the literature, such as the Cornea and Contact Lens Research Unit (CCLRU) Redness Grading Scale developed by the School of Optometry, University of New South Wales. See, for example, Terry et al. in Optom. Vis. Sci. (1993) vol. 70, pages 234-243; and Pult et al. in Ophthal. Physiol. Opt. (2008) vol. 28, pages 13-20. The CCLRU Redness Grading Scale evaluates eye redness on a four-point scale: (0) no eye redness, (1) very slight eye redness, (2) slight eye redness, (3) moderate eye redness, and (4) severe eye redness. See FIG. 1 for an illustration of the eye redness scale.

In certain embodiments, the patient experiences an increase in eye redness of no more than two grades measured using the CCLRU Redness Grading Scale due to the method. In certain embodiments, the patient experiences an increase in eye redness of no more than one grade measured using the CCLRU Redness Grading Scale due to the method.

### Route of Administration

Methods may be further characterized according to the route of administration of the alpha-adrenergic antagonist and/or any muscarinic acetylcholine receptor agonist. For example, in certain embodiments, the alpha-adrenergic antagonist is topically administered to the eye of the patient. In certain embodiments, the alpha-adrenergic antagonist is topically administered to the eye of the patient in the form of an eye drop.

In certain embodiments, the muscarinic acetylcholine receptor agonist is topically administered to the eye of the patient. In certain embodiments, the muscarinic acetylcholine receptor agonist is topically administered to the eye of the patient in the form of an eye drop.

### Time of Administration

Methods may be further characterized according to the time of day when the alpha-adrenergic antagonist is administered to the patient. In certain embodiments, the the alpha-adrenergic antagonist is administered to the eye of the patient at or near the patient's bedtime. In certain embodiments, the dosage of alpha-adrenergic antagonist is administered within 2 hours, 1.5 hours, 1 hour, 45 minutes, 30 minutes, or 15 minutes of the patient's bedtime. In certain embodiments, the dosage of alpha-adrenergic antagonist is administered within 1 hour of the patient's bedtime.

In certain embodiments, the alpha-adrenergic antagonist and any muscarinic acetylcholine receptor agonist are administered concurrently to the eye of the patient. In certain embodiments, the alpha-adrenergic antagonist and any muscarinic acetylcholine receptor agonist are administered separately to the eye of the patient.

### Reduction in Intraocular Pressure in the Eye

Methods may be further characterized according to the reduction in intraocular pressure in the eye due to the method. For example, in certain embodiments, the patient experiences at least a 5% reduction in intraocular pressure in the eye due to the method. In certain embodiments, the patient experiences at least a 10% reduction in intraocular pressure in the eye due to the method. In certain embodiments, the patient experiences at least a 15% reduction in intraocular pressure in the eye due to the method. In certain embodiments, the patient experiences at least a 20% reduction in intraocular pressure in the eye due to the method. In certain embodiments, the patient experiences at least a 25% reduction in intraocular pressure in the eye due to the method. In certain embodiments, the patient experiences at least a 30% reduction in intraocular pressure in the eye due to the method. In certain embodiments, the patient experiences at least a 40% reduction in intraocular pressure in the eye due to the method. In certain embodiments, the patient experiences at least a 50% reduction in intraocular pressure in the eye due to the method.

In certain embodiments, the patient experiences at least a 1 mmHg reduction in intraocular pressure in the eye due to the method. In certain embodiments, the patient experiences at least a 2 mmHg reduction in intraocular pressure in the eye due to the method. In certain embodiments, the patient experiences at least a 3 mmHg reduction in intraocular pressure in the eye due to the method. In certain embodiments, the patient experiences at least a 4 mmHg reduction in intraocular pressure in the eye due to the method. In certain embodiments, the patient experiences at least a 5 mmHg reduction in intraocular pressure in the eye due to the method. In certain embodiments, the patient experiences at least a 6 mmHg reduction in intraocular pressure in the eye due to the method. In certain embodiments, the patient experiences at least a 7 mmHg reduction in intraocular pressure in the eye due to the method. In certain embodiments, the patient experiences at least an 8 mmHg reduction in intraocular pressure in the eye due to the method. In certain embodiments, the patient experiences at least an 10 mmHg reduction in intraocular pressure in the eye due to the method. In certain embodiments, the patient experiences at least a 15 mmHg reduction in intraocular pressure in the eye due to the method. In certain embodiments, the patient experiences at least a 20 mmHg reduction in intraocular pressure in the eye due to the method.

In certain embodiments, the patient experiences reduction in intraocular pressure in the eye in the range of from about 1 mmHg to about 5 mmHg due to the method. In certain embodiments, the patient experiences reduction in intraocular pressure in the eye in the range of from about 5 mmHg to about 10 mmHg due to the method. In certain embodiments, the patient experiences reduction in intraocular pressure in the eye in the range of from about 10 mmHg to about 15 mmHg due to the method. In certain embodiments, the patient experiences reduction in intraocular pressure in the eye in the range of from about 4 mmHg to about 8 mmHg due to the method. In certain embodiments, the patient experiences reduction in intraocular pressure in the eye in the range of from about 8 mmHg to about 12 mmHg due to the method. In certain embodiments, the patient experiences reduction in intraocular pressure in the eye in the range of from about 10 mmHg to about 25 mmHg due to the method. In certain embodiments, the patient experiences reduction in intraocular pressure in the eye in the range of from about 15 mmHg to about 25 mmHg due to the method.

### Duration of Reduction in Intraocular Pressure

Methods may be further characterized according to the duration of reduction in intraocular pressure. For example, in certain embodiments, the reduction in intraocular pressure lasts for a duration of at least 6 hours. In certain embodiments, the reduction in intraocular pressure lasts for a duration of at least 12 hours. In certain embodiments, the reduction in intraocular pressure lasts for a duration of at least 24 hours. In certain embodiments, the reduction in intraocular pressure lasts for a duration of at least 2, 3, 4, 5, 6, 7, 8, 9, 10, 14, 21, or 24 hours.

In certain embodiments, the reduction in intraocular pressure lasts for a duration of at least 2 days. In certain embodiments, the reduction in intraocular pressure lasts for a duration of at least 5 days. In certain embodiments, the reduction in intraocular pressure lasts for a duration of at least 7 days. In certain embodiments, the reduction in intraocular pressure lasts for a duration of at least 2, 3, 4, 5, 6, 7, 8, 9, 10, 14, 21, or 28 days.

In certain embodiments, the reduction in intraocular pressure lasts for a duration of from about 5 to about 24 hours. In certain embodiments, the reduction in intraocular pressure lasts for a duration of from about 12 to about 24 hours. In certain embodiments, the reduction in intraocular pressure lasts for a duration of from about 1 day to about 3 days. In certain embodiments, the reduction in intraocular pressure lasts for a duration of from about 3 days to about 5 days. In certain embodiments, the reduction in intraocular pressure lasts for a duration of from about 5 days to about 7 days. In certain embodiments, the reduction in intraocular pressure lasts for a duration of from about 7 days to about 14 days.

Patient Populations that May Derive Particular Benefits from the Therapeutic Methods

Methods may be further characterized according to patient populations that may derive particular benefits from the therapeutic methods. For example, in certain embodiments, the patient's eye has an intraocular pressure greater than about 22 mmHg before the method is performed. In certain embodiments, the patient's eye has an intraocular pressure greater than about 25 mmHg before the method is performed. In certain embodiments, the patient's eye has an intraocular pressure greater than about 30 mmHg before the method is performed. In certain embodiments, the patient's eye has an intraocular pressure greater than about 40 mmHg before the method is performed.

In certain other embodiments, the patient to begin treatment is characterized as having an intraocular pressure in the range of from about 20 mmHg to about 50 mmHg. In certain other embodiments, the patient to begin treatment is characterized as having an intraocular pressure in the range of from about 30 mmHg to about 50 mmHg. In certain other embodiments, the patient to begin treatment is characterized as having an intraocular pressure in the range of from about 25 mmHg to about 30 mmHg.

In certain other embodiments, the patient to begin treatment is characterized as having an intraocular pressure in the range of from about 20 mmHg to about 30 mmHg before the method is performed. In certain embodiments, the patient to begin treatment is characterized as having an intraocular pressure in the range of from about 20 mmHg to about 25 mmHg before the method is performed.

In certain other embodiments, the patient's eye has an intraocular pressure not greater than about 22 mmHg before the method is performed. In certain other embodiments, the patient to begin treatment is characterized as having an intraocular pressure in the range of from about 12 mmHg to about 22 mmHg. In certain other embodiments, the patient to begin treatment is characterized as having an intraocular pressure in the range of from about 15 mmHg to about 22 mmHg. In certain other embodiments, the patient to begin treatment is characterized as having an intraocular pressure in the range of from about 18 mmHg to about 22 mmHg.

In certain other embodiments, the patient to begin treatment is characterized as having an intraocular pressure in the range of from about 17 mmHg to about 36 mmHg, from about 17 mmHg to about 32 mmHg, from about 17 mmHg to about 28 mmHg, from about 17 mmHg to about 26 mmHg, from about 17 mmHg to about 24 mmHg, or from about 17 mmHg to about 22 mmHg. In certain other embodiments, the patient to begin treatment is characterized as having an intraocular pressure in the range of from about 20 mmHg to about 36 mmHg, from about 20 mmHg to about 32 mmHg, from about 20 mmHg to about 28 mmHg, from about 20 mmHg to about 26 mmHg, from about 20 mmHg to about 24 mmHg, or from about 20 mmHg to about 22 mmHg. In certain other embodiments, the patient to begin treatment is characterized as having an intraocular pressure in the range of from about 20 mmHg to about 26 mmHg, from about 20 mmHg to about 25 mmHg, from about 20 mmHg to about 24 mmHg, from about 20 mmHg to about 23 mmHg, or from about 20 mmHg to about 22 mmHg. In certain other embodiments, the patient to begin treatment is characterized as having an intraocular pressure less than about 23, 24, 25, or 26 mmHg

In certain embodiments, the patient is a human. In certain embodiments, the patient is an adult human. In certain embodiments, the patient is a pediatric human.

In certain embodiments, the patient's eye has a narrow angle.

Administration of an Agent that Reduces Eye Redness

Methods may be further characterized according to optionally administering to the eye of a patient an agent that reduces eye redness (e.g., eye redness caused by an alpha-adrenergic antagonist). For example, in certain embodiments, the method further comprises topically administering to the eye of the patient an agent that reduces eye redness. Exemplary agents that reduce eye redness include brimonidine, tetrahydrozoline, oxymetazoline, naphthazoline, or a pharmaceutically acceptable salt thereof, such as LUMIFY® (which is a commercially available ophthalmic solution containing brimonidine tartrate (0.025% w/w)).

In certain embodiments, the method further comprises topically administering to the eye of the patient brimonidine or a pharmaceutically acceptable salt thereof. In certain embodiments, the method further comprises topically administering to the eye of the patient brimonidine tartrate. In certain embodiments, the method further comprises topically administering to the eye of the patient an ophthalmic solution comprising about 0.025% (w/w) brimonidine tartrate.

### II. Administration of Additional Therapeutic Agent

Another aspect of the invention provides for administration of an additional therapeutic agent. Methods described hereinabove may optionally further comprise administering one or more additional therapeutic agents to the patient. Exemplary additional therapeutic agents include, for example:
A prostaglandin analog, such as latanoprost, bimatoprost, travoprost, tafluprost, latanoprostene bunod, or a pharmaceutically acceptable salt thereof;
A beta blocker, such as timolol or a pharmaceutically acceptable salt thereof;
An alpha agonist, such as brimonidine or a pharmaceutically acceptable salt thereof;
A carbonic anhydrase inhibitor, such as dorzolamide, brinzolamide, acetazolamide, methazolamide, or a pharmaceutically acceptable salt thereof; and
A Rho kinase inhibitor, such as netarsudil or a pharmaceutically acceptable salt thereof.

Latanaoprost may be administered in the form of a sterile, isotonic, buffered aqueous solution of latanoprost with a pH of approximately 6.7 and an osmolality of approximately 267 mOsmol/kg, where each 1 mL of the solution contains 50 micrograms of latanoprost. The solution may optionally contain benzalkonium chloride (0.02% w/w), sodium chloride, sodium dihydrogen phosphate monohydrate, and disodium hydrogen phosphate.

Latanaoprost may be administered to the patient according to the procedures described in the XALATAN® prescribing information, which is hereby incorporated by reference. In certain embodiments, a single daily dose of 1.5 micrograms of latanaoprost is administered to the patient's eye. In certain embodiments, a single daily dose in the range of about 0.5 to about 1.0 micrograms, about 1.0 to about 1.5 micrograms, or about 1.5 to about 2.0 micrograms of latanaoprost is administered to the patient's eye.

Timolol may be administered as timolol maleate in the form of an ophthalmic solution. One or two drops per day of a solution that contains on a 1 mL basis 3.4 mg of timolol maleate may be administered to the eye of the patient. Alternatively, one drop per day of a solution that contains on a 1 mL basis 6.8 mg of timolol maleate may be administered to the eye of the patient.

Netarsudil may be administered to the patient in the form of an ophthalmic solution, such as a sterile, isotonic, buffered aqueous solution containing netarsudil dimesylate (0.02% w/w) having a pH of approximately 5 and an osmolality of approximately 295 mOsmol/kg. Each 1 mL of the solution contains 0.28 mg of netarsudil dimesylate. The aqueous solution may contain benzalkonium chloride (e.g., 0.015% w/w), boric acid, and mannitol. Netarsudil dimesylate may be administered to the patient once per day as one eye drop of the sterile, isotonic, buffered aqueous solution containing netarsudil dimesylate (0.02% w/w) having a pH of approximately 5 and an osmolality of approximately 295 mOsmol/kg.

The amount of each therapeutic agent and the relative timing of administration of each therapeutic agent may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like

In certain embodiments, the therapeutic agents may act additively or synergistically. A synergistic combination may allow the use of lower dosages of one or more agents and/or less frequent administration of one or more agents of a combination therapy. A lower dosage or less frequent administration of one or more agents may lower toxicity of the therapy without reducing the efficacy of the therapy.

### III. Implantable Ocular Device

Compositions described herein may be administered to the patient's eye via an implantable ocular device that dispenses the composition. The implantable ocular device may be configured to dispense the composition at a desired rate and/or frequency. In certain embodiments, the implantable ocular device is a slow release insert.

### IV. Ophthalmic Solutions

Therapeutically active agents are desirably administered to the eye of the patient in the form of an ophthalmic solution. Such an ophthalmic solution comprises one or more therapeutically active agents and a pharmaceutically acceptable carrier. Desirably, the ophthalmic solution exhibits good storage stability to permit distribution of the ophthalmic solution through normal distribution channels for pharmaceuticals. In certain embodiments, the pharmaceutically acceptable carrier is water. Additional components may be added to the ophthalmic solution in order to optimize performance properties of the ophthalmic solution. Exemplary additional components include, for example, a chelating agent (e.g., EDTA), polyol compound, poly(C₂₋₄alkylene)glycol polymer, dextran, cellulose agent, buffer, tonicity modifier, preservative, antioxidant, viscosity modifying agent, corneal permeation enhancing agent, solubilizing agent, stabilizing agent, surfactant, demulcent polymer, wetting agent, and other materials.

Ophthalmic solutions may be further characterized according to the presence or absence of one or more of a chelating agent (e.g., EDTA), polyol compound, poly(C₂₋₄alkylene)glycol polymer, dextran, cellulose agent, buffer, tonicity modifier, preservative, antioxidant, viscosity modifying agent, corneal permeation enhancing agent, solubilizing agent, stabilizing agent, surfactant, demulcent polymer, wetting agent, and other materials. In certain embodiments, the ophthalmic solution does not contain a chelating agent (e.g., EDTA). In certain embodiments, the ophthalmic solution does not contain a preservative.

Various therapeutic methods above involve administering a dosage of phentolamine or a pharmaceutically acceptable salt thereof to the patient. The dosage of phentolamine or a pharmaceutically acceptable salt thereof is desirably in the form of an ophthalmic solution. The ophthalmic solution is formulated to be suitable for administration to the eye of a patient, and desirably provides immediate release of phentolamine, that is, the ophthalmic solution is not a sustained release formulation that delivers phentolamine over an extended duration, such as hours, days or weeks.

The ophthalmic solution desirably comprises an aqueous pharmaceutically acceptable carrier and phentolamine or a pharmaceutically acceptable salt thereof. The ophthalmic solution may contain excipients(s) that are suitable for administration to the eye. Various pharmaceutically acceptable salts are described in the literature. The preferred salt form of phentolamine is phentolamine mesylate. Accordingly, the methods may use an ophthalmic solution that comprises an aqueous pharmaceutically acceptable carrier and phentolamine mesylate.

Accordingly, in certain embodiments, the dosage utilized in the methods is an ophthalmic solution comprising an aqueous pharmaceutically acceptable carrier and phentolamine or a pharmaceutically acceptable salt thereof. In certain other embodiments, the dosage is an ophthalmic solution comprising an aqueous pharmaceutically acceptable carrier and phentolamine mesylate. In certain other embodiments, the dosage is an ophthalmic solution comprising water, a polyol, and phentolamine or a pharmaceutically acceptable salt thereof. In certain other embodiments, the dosage is an ophthalmic solution comprising water, mannitol, and phentolamine mesylate. In certain other embodiments, the dosage is an ophthalmic solution comprising water, a polyol, an alkali metal carboxylate, and phentolamine or a pharmaceutically acceptable salt thereof. In certain other embodiments, the dosage is an ophthalmic solution comprising water, mannitol, sodium acetate, and phentolamine mesylate.

Other ophthalmic solutions that are contemplated for use in the present invention include, for example, (i) aqueous ophthalmic solutions free of a chelating agent, and (ii) polyvinylpyrrolidone artificial tears formulations, each of which are described in more detail below.

Ophthalmic solutions may be further characterized according to the viscosity of the solution. In certain embodiments, the ophthalmic solution at a temperature of about 25° C. has a viscosity in the range of 0.9 cP to about 1.1 cP. In certain embodiments, the ophthalmic solution at a temperature of about 25° C. has a viscosity of about 1 cP.

Ophthalmic solutions may be further characterized according to rate of release of any active ingredients. For example, in certain embodiments, the ophthalmic solution is an immediate release ophthalmic solution. In certain embodiments, the ophthalmic solution is an extended release ophthalmic solution.

### Aqueous Ophthalmic Solution Free of a Chelating Agent

In certain embodiments, the dosage utilized in the methods is an aqueous ophthalmic solution free of a chelating agent, wherein said solution comprises (a) phentolamine or a pharmaceutically acceptable salt thereof; (b) at least one polyol compound, such as a polyol compound having a molecular weight less than 250 g/mol; (c) at least one buffer; and (d) water; wherein the solution does not contain a chelating agent. The amount of ingredients in the aqueous ophthalmic solutions may be selected in order to achieve particular performance properties, such as stability to storage, minimize irritation to the eye of a patient, and enhance penetration of phentolamine into the eye of a patient.

One exemplary preferred solution is an aqueous ophthalmic solution free of a chelating agent comprising: (a) about 0.1% (w/v) to about 4% (w/v) of phentolamine or a pharmaceutically acceptable salt thereof, (b) about 1% (w/v) to about 6% (w/v) of at least one polyol compound having a molecular weight less than 250 g/mol; (c) about 0.1 mM to about 10 mM of at least one buffer; and (d) water; wherein the solution has a pH in the range of 4.0 to 7.5 and does not contain a chelating agent.

Exemplary components and features of the aqueous ophthalmic solutions are described in more detail below.

### Phentolamine & Pharmaceutically Acceptable Salts

The aqueous ophthalmic solution comprises phentolamine or a pharmaceutically acceptable salt of phentolamine. Exemplary pharmaceutically acceptable salts include, for example, the hydrochloric acid salt and mesylate salt. Accordingly, in certain embodiments, the solution comprises phentolamine (i.e., as the free base). In certain other embodiments, the solution comprises phentolamine hydrochloride. In certain yet other embodiments, the solution comprises phentolamine mesylate.

The amount of phentolamine or a pharmaceutically acceptable salt thereof in the aqueous ophthalmic solution may be adjusted in order to achieve desired performance properties. For example, where is it desired to provide a larger amount of phentolamine (or pharmaceutically acceptable salt thereof) to the patient in a single administration of the aqueous ophthalmic solution, the concentration of phentolamine (or pharmaceutically acceptable salt thereof) is increased in the aqueous ophthalmic solution. Single administration of aqueous ophthalmic solutions having a higher concentration of phentolamine (or pharmaceutically acceptable salt thereof) may provide the patient with reduced intraocular pressure for a longer duration of time because more phentolamine (or pharmaceutically acceptable salt thereof) is administered to the patient.

Accordingly, in certain embodiments, the aqueous ophthalmic solution comprises from about 0.1% (w/v) to about 5% (w/v) of phentolamine or a pharmaceutically acceptable salt thereof. In certain embodiments, the aqueous ophthalmic solution comprises from about 0.1% (w/v) to about 1% (w/v) of phentolamine or a pharmaceutically acceptable salt thereof, about 1% (w/v) to about 2% (w/v) of phentolamine or a pharmaceutically acceptable salt thereof, about 2% (w/v) to about 3% (w/v) of phentolamine or a pharmaceutically acceptable salt thereof, about 3% (w/v) to about 4% (w/v) of phentolamine or a pharmaceutically acceptable salt thereof, about 4% (w/v) to about 5% (w/v) of phentolamine or a pharmaceutically acceptable salt thereof. In certain embodiments, the aqueous ophthalmic solution comprises from about 0.1% (w/v) to about 2% (w/v) of phentolamine or a pharmaceutically acceptable salt thereof. In certain embodiments, the aqueous ophthalmic solution comprises from about 0.25% (w/v) to about 2% (w/v) of phentolamine or a pharmaceutically acceptable salt thereof. In certain other embodiments, the aqueous ophthalmic solution comprises from about 0.5% (w/v) to about 2% (w/v) of phentolamine or a pharmaceutically acceptable salt thereof. In certain other embodiments, the aqueous ophthalmic solution comprises from about 0.25% (w/v) to about 1% (w/v) of phentolamine or a pharmaceutically acceptable salt thereof. In certain other embodiments, the aqueous ophthalmic solution comprises about 1% (w/v) of phentolamine or a pharmaceutically acceptable salt thereof. In certain other embodiments, the aqueous ophthalmic solution comprises from about 0.1% (w/v) to about 4% (w/v) of phentolamine mesylate. In certain other embodiments, the aqueous ophthalmic solution comprises from about 0.1% (w/v) to about 2% (w/v) of phentolamine mesylate. In certain other embodiments, the aqueous ophthalmic solution comprises from about 0.25% (w/v) to about 2% (w/v) of phentolamine mesylate. In certain other embodiments, the aqueous ophthalmic solution comprises from about 0.5% (w/v) to about 2% (w/v) of phentolamine mesylate. In certain other embodiments, the aqueous ophthalmic solution comprises from about 0.25% (w/v) to about 1% (w/v) of phentolamine mesylate. In certain other embodiments, the aqueous ophthalmic solution comprises about 1% (w/v) of phentolamine mesylate. In certain other embodiments, the aqueous ophthalmic solution comprises about 0.25% (w/v) or about 0.5% (w/v) of phentolamine mesylate.

### Polyol Compounds

The aqueous ophthalmic solution comprises one or more polyol compounds. The polyol compound is an organic compound having at least two hydroxyl groups (e.g., from 2 to about 6 hydroxyl groups). The polyol compound is beneficial to the aqueous ophthalmic solution because, for example, it can increase the stability of the aqueous ophthalmic solution to storage and/or modify the tonicity of the aqueous ophthalmic solution. Exemplary polyol compounds include, for example, mannitol, glycerol, propylene glycol, ethylene glycol, sorbitol, and xylitol.

The aqueous ophthalmic solution may contain a single polyol compound or a mixture of one or more polyol compounds. In other words, the aqueous ophthalmic solution comprises at least one polyol compound. In certain embodiments, the aqueous ophthalmic solution comprises at least one polyol compound that is mannitol, glycerol, propylene glycol, ethylene glycol, sorbitol, or xylitol. In certain other embodiments, the at least one polyol compound is mannitol. In certain other embodiments, the at least one polyol compound is glycerol. In certain other embodiments, the at least one polyol compound is propylene glycol. In certain other embodiments, the at least one polyol compound is mannitol, and the solution further comprises glycerol. In certain other embodiments, the at least one polyol compound is mannitol, and the solution further comprises propylene glycol. In certain other embodiments, the at least one polyol compound is glycerol, and the solution further comprises propylene glycol. In certain other embodiments, the mannitol described in embodiments above is D-mannitol.

The amount of the at least one polyol compound in the aqueous ophthalmic solution may be selected in order to achieve desired performance properties for the solution. The polyol compound may, for example, increase the stability of the solution to storage and/or modify the tonicity of the solution to make it more suitable for administration to the eye of a patient. In certain embodiments, the aqueous ophthalmic solution comprises from about 2% (w/v) to about 5% (w/v) of the at least one polyol compound. In certain other embodiments, the aqueous ophthalmic solution comprises from about 3.5% (w/v) to about 4.5% (w/v) of the at least one polyol compound. In certain other embodiments, the aqueous ophthalmic solution comprises about 4% (w/v) of the at least one polyol compound. In certain other embodiments, the aqueous ophthalmic solution comprises from about 2% (w/v) to about 3% (w/v) mannitol, and about 0.5% (w/v) to about 1.5% (w/v) glycerin. In certain other embodiments, the mannitol described in embodiments above is D-mannitol.

In certain embodiments, the amount of polyol may be selected based on the amount of phentolamine (or pharmaceutically acceptable salt thereof), such that there is an inverse relationship between the amount of phentolamine (or pharmaceutically acceptable salt thereof) and the polyol in order to achieve isotonicity with the eye. For example, in embodiments where the aqueous ophthalmic solution contains about 2% (w/v) phentolamine, mannitol is present in the solution at a concentration of about 3% (w/v). In embodiments where the aqueous ophthalmic solution contains about 1% (w/v) phentolamine, mannitol is present in the solution at a concentration of about 4% (w/v). To further illustrate this principle, in embodiments where the aqueous ophthalmic solution contains about 0.5% (w/v) phentolamine, mannitol may be present in the solution at a concentration of about 4.5% (w/v). In certain embodiments, the mannitol described in embodiments above is D-mannitol.

It is appreciated that the aqueous ophthalmic solution can contain additional ingredients described herein, such as various polymer materials. One such embodiment is an aqueous ophthalmic solution comprising, for example, at least one polyol compound that is propylene glycol, and further comprising polypropylene glycol, such as polypropylene glycol having a weight average molecular weight in the range of about 5,000 g/mol to about 100,000 g/mol.

### Poly(C₂₋₄alkylene)glycol Polymer

The aqueous ophthalmic solution may optionally comprise a poly(C₂₋₄alkylene)glycol polymer. An exemplary poly(C₂₋₄alkylene)glycol polymer is polypropylene glycol, such as a polypropylene glycol having a weight average molecular weight in the range of about 5,000 g/mol to about 100,000 g/mol, about 10,000 g/mol to about 50,000 g/mol, or about 50,000 g/mol to about 100,000 g/mol.

### Dextran

The aqueous ophthalmic solution may optionally comprise dextran. Dextran is a commercially available, branched polysaccharide comprising glucose molecules. The amount of dextran in the aqueous ophthalmic solution may be selected to achieve certain performance properties. In certain embodiments, the aqueous ophthalmic solution comprises from about 0.01% (w/v) to about 2% (w/v) dextran. In certain other embodiments, the aqueous ophthalmic solution comprises from about 0.01% (w/v) to about 1% (w/v) dextran.

The dextran may be further characterized according to its weight average molecular weight. In certain embodiments, the dextran has a weight average molecular weight in the range of about 65,000 g/mol to about 75,000 g/mol. In certain other embodiments, the dextran has a weight average molecular weight of about 70,000 g/mol. In yet other embodiments, the dextran has a weight average molecular weight in the range of about 5,000 g/mol to about 100,000 g/mol, about 10,000 g/mol to about 50,000 g/mol, or about 50,000 g/mol to about 100,000 g/mol.

### Cellulose Agent

The aqueous ophthalmic solution may optionally comprise a cellulose agent. Exemplary cellulose agents include, for example, cellulose, carboxymethyl cellulose, hydroxyethylcellulose, hydroxpropylcellulose, and hydroxypropylmethyl cellulose. In certain embodiments, the cellulose agent is hydroxypropylmethyl cellulose. In certain other embodiments, the cellulose agent is cellulose, carboxymethyl cellulose, hydroxyethylcellulose, or hydroxpropylcellulose. The amount of cellulose agent in the aqueous ophthalmic solution may be selected in order to achieve desired performance properties. For example, in certain embodiments, the aqueous ophthalmic solution comprises from about 0.01% (w/v) to about 2% (w/v) cellulose agent.

The cellulose agent may be further characterized according to its weight average molecular weight. In certain embodiments, the cellulose agent has a weight average molecular weight in the range of about 5,000 g/mol to about 100,000 g/mol, about 10,000 g/mol to about 50,000 g/mol, or about 50,000 g/mol to about 100,000 g/mol.

### Buffer

The aqueous ophthalmic solution comprises at least one buffer. The buffer imparts to the solution a buffering capacity, that is, the capacity to neutralize, within limits, either acids or bases (alkali) with relatively little or no change in the original pH. The buffer may be an acid, a base, or a combination of an acid and a base. The buffer may be organic, inorganic, or a combination of organic and inorganic components. It should be understood that the buffer at least partially dissociates in aqueous solution to form a mixture of, e.g., an acid and conjugate base or a base and conjugate acid. For example, the buffer may be a combination of a carboxylic acid and its carboxylate salt (e.g., a combination of acetic acid and sodium acetate). In another embodiment, the buffer may be a combination of an acid and a base, where the acid and the base are not conjugates. For example, the acid may be boric acid and the base may be tris(hydroxymethyl)aminomethane (TRIS).

Exemplary buffers include organic acids (e.g., acetic acid, sorbic acid, and oxalic acid), a borate salt, a hydrogen carbonate salt, a carbonate salt, a gluconate salt, a lactate salt, a phosphate salt, a propionate salt, a perborate salt, tris-(hydroxymethyl)aminomethane (TRIS), bis(2-hydroxyethyl)-imino-tris-(hydroxymethyl)aminoalcohol (bis-tris), N-[2-hydroxy-1,1-bis(hydroxymethyl)ethyl]glycine (tricene), N-[2-hydroxy-1,1-bis(hydroxymethyl)ethyl]glycine, 3-(N-morpholino)propanesulfonic acid, N-(carbamoylmethyl)taurine (ACES), an amino acid, salts thereof, and combinations thereof. It should be understood that the salt form of a buffer may comprise any suitable counterion. For example, the salt form of an acid may comprise an alkali or alkaline earth metal counterion.

The buffer can be characterized according to its strength, i.e., the buffering capacity. The buffering capacity can be tested, for example, by determining the millimoles (mM) of strong acid or base (or respectively, hydrogen or hydroxide ions) required to change the pH of a buffer solution by one unit when added to one liter (a standard unit) of the buffer solution. The buffering capacity generally depends on the type and concentration of the buffer components and can be greater in particular pH ranges. For example, a buffer may have an optimal buffering capacity in a pH range near the pKₐ of the buffer, e.g., within about 1 pH unit or within about 2 pH units of the pKₐ the buffer. In certain embodiments, the buffer is a weak buffer, such as an alkali metal carboxylate (e.g., sodium acetate).

In certain embodiments, the buffer is a weak acid buffer having one or more of the following characteristics: (a) a pKₐ of from about 4.0 to about 6.0; more preferably, from about 4.5 to about 5.5; and (b) a lipophilicity value Log P of from about −0.50 to about 1.5; more preferably, from about −0.25 to about 1.35.

The amount of buffer can be adjusted in order to achieve desired performance properties for the aqueous ophthalmic solution. For example, in certain embodiments, the buffer may be present at a concentration of less than about 10 mM, less than about 7 mM, less than about 5 mM, less than about 3 mM, or less than about 2 mM. In some embodiments, the buffer may be present at a concentration of from about 1 mM to about 10 mM, from about 1 mM to about 7 mM, from about 1 mM to about 5 mM, from about 1 mM to about 3 mM, from about 1 mM to about 2 mM, from about 2 mM to about 5 mM, or from about 2 mM to about 3 mM. In yet other embodiments, the buffer is present at a concentration of about 3 mM.

The amount and identity of the buffer may be selected in order to achieve certain performance properties for the aqueous ophthalmic solution. For example, the amount of buffer may impact the quantity of acid that may be neutralized before there is substantial change in the pH of the aqueous ophthalmic solution. Also, the amount of buffer may impact the tonicity of the aqueous ophthalmic solution. Desirably, the quantity and identity of the buffer should be selected in order to minimize any irritation that may be caused by administration of the aqueous ophthalmic solution to the eye of a patient. Accordingly, in certain embodiments, the buffer is present at a concentration in the range of about 2 mM to about 4 mM. In yet other embodiments, the buffer is present at a concentration of about 3 mM. In certain embodiments, the buffer comprises an alkali metal alkylcarboxylate. In certain other embodiments, the buffer comprises an alkali metal acetate. In yet other embodiments, the buffer comprises sodium acetate.

### Solution pH

The aqueous ophthalmic solution may be characterized according to the pH of the solution. Desirably, the aqueous ophthalmic solution has a pH in the range of 4.0 to 7.5. In certain embodiments, the aqueous ophthalmic solution has a pH in the range of 4.5 to 7.5. In certain embodiments, the solution has a pH in the range of 4.5 to 6.0. In certain other embodiments, the solution has a pH in the range of 4.5 to 5.5. In yet other embodiments, the solution has a pH in the range of 4.7 to 5.1.

### Additional Materials for Aqueous Ophthalmic Solutions

The aqueous ophthalmic solutions may contain additional materials in order to make the composition more suitable for administration to the eye of a patient. Exemplary additional materials are described below and include, for example, a tonicity modifier, preservative, antioxidant, viscosity modifying agent, stabilizing agent, corneal permeation enhancing agent, and surfactants.

### A. Tonicity Modifier

The aqueous ophthalmic solution may optionally comprise one or more tonicity modifiers. The tonicity modifier may be ionic or non-ionic. In certain embodiments, the tonicity modifier may be a salt, a carbohydrate, or a polyol. Exemplary tonicity modifiers include alkali metal or alkaline earth metal halides (such as LiBr, LiCl, LiI, KBr, KCl, KI, NaBr, NaCl, NaI, CaCl₂, and MgCl₂), boric acid, dextran (e.g., Dextran 70), cyclodextrin, dextrose, mannitol, glycerin, urea, sorbitol, propylene glycol, or a combination thereof.

It is appreciated that the tonicity modifier may be added to the aqueous ophthalmic solution in an amount sufficient to provide a desired osmolality. In certain embodiments, the tonicity modifier is present in the aqueous ophthalmic solution in an amount sufficient so that the aqueous ophthalmic solution has an osmolality ranging from about 50 to about 1000 mOsm/kg, from about 100 to about 400 mOsm/kg, from about 200 to about 400 mOsm/kg, or from about 280 to about 380 mOsm/kg. In certain embodiments, a tonicity modifier may be present in an amount ranging from about 0.01% (w/v) to about 7% (w/v), about 0.01% (w/v) to about 5% (w/v), about 0.01% (w/v) to about 1% (w/v), about 0.1% (w/v) to about 1% (w/v), about 0.05% (w/v) to about 5% (w/v), about 0.05% (w/v) to about 0.5% (w/v), about 1% (w/v) to about 3% (w/v), or about 2% (w/v) to about 4% (w/v), of the aqueous ophthalmic solution.

### B. Preservative

The aqueous ophthalmic solution may optionally comprise one or more preservatives in order to, for example, reduce or prevent microbial contamination. Exemplary preservatives include quaternary ammonium salts such as polyquaternium-1, cetrimide, benzalkonium chloride, or benzoxonium chloride; alkyl-mercury salts of thiosalicylic acid such as thiomersal, phenylmercuric nitrate, phenylmercuric acetate, or phenylmercuric borate; parabens such as methylparaben or propylparaben; alcohols such as chlorobutanol, benzyl alcohol, phenyl ethanol, cyclohexanol, 3-pentanol, or resorcinol; a peroxide; chlorine dioxide or PURITE; guanidine derivatives such as chlorohexidine gluconate or polyaminopropyl biguanide; and combinations thereof.

The amount of preservative can be adjusted in order to achieve desired performance properties for the aqueous ophthalmic solution. In certain embodiments, the preservative is present in an amount less than about 5% (w/v), 3% (w/v), 1% (w/v), or 0.1% (w/v) of the aqueous ophthalmic solution. In certain other embodiments, the preservative is present in an amount ranging from about 0.01% (w/v) to about 5% (w/v), about 0.01% (w/v) to about 1% (w/v), about 0.1% (w/v) to about 1% (w/v), about 0.05% (w/v) to about 5% (w/v), or about 0.05% (w/v) to about 0.5% (w/v), of the aqueous ophthalmic solution.

### C. Antioxidant

The aqueous ophthalmic solution may optionally comprise one or more antioxidants. Exemplary antioxidants for use in the aqueous ophthalmic solutions described herein include water soluble antioxidants such as ascorbic acid, cysteine hydrochloride, sodium bisulfate, sodium metabisulfite, sodium bisulfite, sodium sulfite, and the like; and oil-soluble antioxidants, such as ascorbyl palmitate, butylated hydroxyanisole (BHA), butylated hydroxytoluene (BHT), lecithin, propyl gallate, alpha-tocopherol, and the like.

The amount of antioxidant can be adjusted in order to achieve desired performance properties for the aqueous ophthalmic solution. In certain embodiments, the antioxidant is present in an amount less than about 5% (w/v), 3% (w/v), 1% (w/v), or 0.1% (w/v) of the aqueous ophthalmic solution. In certain other embodiments, the antioxidant is present in an amount ranging from about 0.01% (w/v) to about 5% (w/v), about 0.01% (w/v) to about 1% (w/v), about 0.1% (w/v) to about 1% (w/v), about 0.05% (w/v) to about 5% (w/v), or about 0.05% (w/v) to about 0.5% (w/v), of the aqueous ophthalmic solution.

### D. Viscosity Modifying Agent

The aqueous ophthalmic solution may optionally comprise one or more viscosity modifying agents. The viscosity modifying agent may be used, for example, to increase the absorption of an active agent or increase the retention time of the aqueous ophthalmic solution in the eye. Exemplary viscosity modifying agents include polyvinylpyrrolidone, methylcellulose, hydroxypropyl methylcellulose, hydroxyethylcellulose, hydroxpropylcellulose, carboxymethylcellulose (CMC) and salts thereof (e.g., CMC sodium salt), gelatin, cellulose glycolate, sorbitol, niacinamide, an alpha-cyclodextran, polyvinyl alcohol, polyethylene glycol, hyaluronic acid, a polysaccharide, a monosaccharide, and combinations thereof.

The amount of viscosity modifying agent can be adjusted in order to achieve desired performance properties for the aqueous ophthalmic solution. In certain embodiments, the viscosity modifying agent is present in an amount less than about 10% (w/v), 5% (w/v), 3% (w/v), 1% (w/v), or 0.1% (w/v) of the aqueous ophthalmic solution. In certain other embodiments, the viscosity modifying agent is present in an amount ranging from about 0.01% (w/v) to about 5% (w/v), about 0.01% (w/v) to about 1% (w/v), about 0.1% (w/v) to about 1% (w/v), about 0.05% (w/v) to about 5% (w/v), or about 0.05% (w/v) to about 0.5% (w/v), of the aqueous ophthalmic solution. In certain other embodiments, the viscosity modifying agent is present in an amount sufficient to provide an aqueous ophthalmic solution with a viscosity in the range of about 30 centipoise to about 100 centipoise.

The viscosity modifying agent may be a polymer that results in delayed release of one or more therapeutic agents in the solution. The identity of the polymer may be selected so as to achieve a desired time-release profile for the one or more therapeutic agents.

### E. Corneal Permeation Enhancing Agent

The aqueous ophthalmic solution may optionally comprise one or more agents for enhancing corneal permeation of phentolamine (or a pharmaceutically acceptable salt thereof). Exemplary agents for enhancing corneal permeation include polymers, organic acids, esters of an organic acid (e.g., a monoglyceride of fatty acid having 8 to 12 carbon atoms), cyclodextrin, benzalkonium chloride (BAK), EDTA, caprylic acid, citric acid, boric acid, sorbic acid, polyoxyethylene-20-stearyl ether (PSE), polyethoxylated castor oil (PCO), deoxycholic acid sodium salt (DC), cetylpyridinium chloride (CPC), laurocapram, hexamethylenelauramide, hexamethyleneoctanamide, decylmethylsulfoxide, methyl sulfone, dimethyl sulfoxide, and combinations thereof.

The amount of corneal permeation enhancing agent can be adjusted in order to achieve desired performance properties for the aqueous ophthalmic solution. In certain embodiments, the corneal permeation enhancing agent is present in an amount less than about 10% (w/v), 5% (w/v), 1% (w/v), or 0.1% (w/v) of the aqueous ophthalmic solution. In certain other embodiments, the corneal permeation enhancing agent is present in an amount ranging from about 0.01% (w/v) to about 5% (w/v), about 0.01% (w/v) to about 1% (w/v), about 0.1% (w/v) to about 1% (w/v), about 0.05% (w/v) to about 5% (w/v), about 0.05% (w/v) to about 0.5% (w/v), about 1% (w/v) to about 3% (w/v), or about 2% (w/v) to about 4% (w/v), of the aqueous ophthalmic solution.

### F. Solubilizing Agent

The aqueous ophthalmic solution may optionally comprise one or more solubilizing agents to improve the solubility of phentolamine (or a pharmaceutically acceptable salt thereof) in the aqueous ophthalmic solution. Exemplary solubilizing agents include, for example, a fatty acid glycerol poly-lower alkylene (i.e., a C₁ to C₇, linear or branched) glycol ester, fatty acid poly-lower alkylene glycol ester, polyalkylene glycol (e.g., polyethylene glycol), glycerol ether of vitamin E, tocopherol polyethylene glycol 1000 succinate (TPGS), tyloxapol, polysorbate 20, polysorbate 40, polysorbate 60, polysorbate 80, polyoxyethylene/polyoxypropylene surfactants (e.g., Pluronic F-68, F-84 and P-103), cyclodextrin, and combinations thereof.

The amount of solubilizing agent can be adjusted in order to achieve desired performance properties for the aqueous ophthalmic solution. In certain embodiments, the solubilizing agent is present in an amount less than about 10% (w/v), 5% (w/v), 3% (w/v), 1% (w/v), or 0.10% (w/v) of the aqueous ophthalmic solution. In certain other embodiments, the solubilizing agent is present in an amount ranging from about 0.01% (w/v) to about 5% (w/v), about 0.01% (w/v) to about 1% (w/v), about 0.10% (w/v) to about 1% (w/v), about 0.05% (w/v) to about 5% (w/v), or about 0.05% (w/v) to about 0.5% (w/v), of the aqueous ophthalmic solution.

### G. Stabilizing Agent

The aqueous ophthalmic solution may optionally comprise one or more stabilizing agents in order to improve the stability of the aqueous ophthalmic solution to storage, etc. Stabilizing agents described in the pharmaceutical literature are contemplated to be amenable for use in the aqueous ophthalmic solutions described herein. Exemplary stabilizing agents include an alcohol (e.g., polyols, such as mannitol, glycerol, propylene glycol, sorbitol, and xylitol), polyalkylene glycols such as polyethylene glycol, polypropylene glycol, polyethylene glycol-nonphenol ether, polyethylene glycol sorbitan monolaurate, polyethylene glycol sorbitan monooleate, polyethylene glycol sorbitan monooleate, polyethylene glycol sterarate, polyethylene glycol polypropylene glycol ether, polyvinyl alcohol, polyvinyl pyrrolidine, ascorbic acid, vitamin E, N-acetylcarnosine (NAC), sorbic acid, and combinations thereof. In certain embodiments, the stabilizing agent is a polymer, such as one of the polymers mentioned above.

The amount of stabilizing agent can be adjusted in order to achieve desired performance properties for the aqueous ophthalmic solution. In certain embodiments, the stabilizing agent is present in an amount less than about 10% (w/v), 5% (w/v), or 1% (w/v) of the aqueous ophthalmic solution. In certain other embodiments, the stabilizing agent is present in an amount ranging from about 0.01% (w/v) to about 5% (w/v), about 0.01% (w/v) to about 1% (w/v), or about 0.01% (w/v) to about 0.10% (w/v) of the aqueous ophthalmic solution.

### H. Surfactant

The aqueous ophthalmic solution may optionally comprise one or more surfactants. Exemplary surfactants include Polysorbate 20 (i.e., polyoxyethylene (20) sorbitan monolaurate), Polysorbate 40 (i.e., polyoxyethylene (20) sorbitan monopalmitate), Polysorbate 60 (i.e., polyoxyethylene (20) sorbitan monostearate), Polysorbate 80 (i.e., polyoxyethylene (20) sorbitan monooleate), glyceryl stearate, isopropyl stearate, polyoxyl stearate, propylene glycol stearate, sucrose stearate, polyethylene glycol, a polypropylene oxide, a polypropylene oxide copolymer, Pluronic F68, Pluronic F-84, Pluronic P-103, an alcohol ethoxylate, an alkylphenol ethoxylate, an alkyl glycoside, an alkyl polyglycoside, a fatty alcohol, hydroxypropylmethyl cellulose (HPMC), carboxymethyl cellulose (CMC), cyclodextrin, a polyacrylic acid, phosphatidyl chloline, phosphatidyl serine, and combinations thereof.

The amount of surfactant can be adjusted in order to achieve desired performance properties for the aqueous ophthalmic solution. In certain embodiments, the surfactant is present in an amount less than about 10% (w/v), 5% (w/v), 3% (w/v), 1% (w/v), or 0.1% (w/v) of the aqueous ophthalmic solution. In certain other embodiments, the surfactant is present in an amount ranging from about 0.01% (w/v) to about 5% (w/v), about 0.01% (w/v) to about 1% (w/v), about 0.1% (w/v) to about 1% (w/v), about 0.05% (w/v) to about 5% (w/v), or about 0.05% (w/v) to about 0.5% (w/v), of the aqueous ophthalmic solution.

### I. Demulcent Polymers

The aqueous ophthalmic solution may optionally comprise one or more demulcent polymers. Because of their ability to hold large amounts of water, demulcent polymers are useful for coating and moisturizing the cornea of the eye. Exemplary demulcent polymers include cellulose derivatives, dextran 40, dextran 70, gelatin, and liquid polyols.

### J. Wetting Agents

The aqueous ophthalmic solution may optionally comprise one or more wetting agents. Wetting agents can be used to wet the surface of the eye. Exemplary wetting agents include polysorbates, poloxamers, tyloxapol, and lecithin.

### K. Additional Materials

The aqueous ophthalmic solutions may optionally comprise one or more additional materials, such as acetylcysteine, cysteine, sodium hydrogen sulfite, butyl-hydroxyanisole, butyl-hydroxytoluene, alpha-tocopherol acetate, thiourea, thiosorbitol, sodium dioctyl sulfosuccinate, monothioglycerol, lauric acid sorbitol ester, triethanol amine oleate, or palmitic acid esters.

Further, the aqueous ophthalmic solutions may comprise a carrier, such as one or more of the exemplary carriers are described in for example, Martin, Remington's Pharmaceutical Sciences, 15th Ed., Mack Publ. Co., Easton, PA [1975]). The carrier can be, for example, a mixture of water and a water-miscible solvent (e.g., an alcohol such as glycerin, a vegetable oil, or a mineral oil). Other exemplary carriers include a mixture of water and one or more of the following materials: hydroxyethylcellulose, carboxymethylcellulose, methylcellulose, an alkali metal salt of carboxymethylcellulose, hydroxymethylcellulose, methylhydroxypropylcellulose, hydroxypropylcellulose, ethyl oleate, polyvinylpyrrolidone, an acrylate polymer, a methacrylate polymer, a polyacrylamide, gelatin, an alginate, a pectin, tragacanth, karaya gum, xanthan gum, carrageenin, agar, acacia, a starch (such as starch acetate or hydroxypropyl starch), polyvinyl alcohol, polyvinyl methyl ether, polyethylene oxide, or a cross-linked polyacrylic acid.

### Exemplary Aqueous Ophthalmic Solutions

The aqueous ophthalmic solutions having been generally described above will now be more specifically described by reference to the following more specific examples. The following more specific examples are only exemplary and are not intended to limit the scope of the invention in any way.

One such exemplary solution is an aqueous ophthalmic solution free of a chelating agent comprising: (a) about 0.1% (w/v) to about 2% (w/v) of phentolamine mesylate; (b) about 1% (w/v) to about 6% (w/v) of at least one polyol compound selected from the group consisting of is mannitol, glycerol, and propylene glycol; (c) about 1 mM to about 6 mM of an alkali metal acetate; and (d) water; wherein the solution has a pH in the range of 4 to 6 and does not contain a chelating agent.

The aqueous ophthalmic solution may be more specifically defined according to the following embodiments. For example, in certain embodiments, the aqueous ophthalmic solution comprises from about 0.25% (w/v) to about 1% (w/v) of phentolamine mesylate. In certain embodiments, the aqueous ophthalmic solution comprises from about 1% (w/v) to about 4% (w/v) mannitol. In certain other embodiments, the aqueous ophthalmic solution comprises 4% (w/v) mannitol. In certain embodiments, the alkali metal acetate is sodium acetate. In certain other embodiments, the aqueous ophthalmic solution comprises 3 mM sodium acetate. In still other embodiments, the aqueous ophthalmic solution consists of (i) about 0.25% (w/v) to about 1% (w/v) of phentolamine mesylate; (ii) about 1% (w/v) to about 6% (w/v) of one or more polyol compounds selected from the group consisting of mannitol, glycerol, and propylene glycol; (iii) about 1 mM to about 6 mM of an alkali metal acetate; (iv) acetic acid; and (v) water; wherein the solution has a pH in the range of 4 to 6.

Another such exemplary solution is an aqueous ophthalmic solution free of a chelating agent comprising: (a) about 0.5% (w/v) to about 2% (w/v) of phentolamine mesylate; (b) about 1% (w/v) to about 6% (w/v) of at least one polyol compound selected from the group consisting of is mannitol, glycerol, and propylene glycol; (c) about 1 mM to about 6 mM of an alkali metal acetate; and (d) water; wherein the solution has a pH in the range of 4.5 to 5.5 and does not contain a chelating agent.

The aqueous ophthalmic solution may be more specifically defined according to the following embodiments. For example, in certain embodiments, the at least one polyol is mannitol. In certain embodiments, the aqueous ophthalmic solution comprises from about 1% (w/v) to about 4% (w/v) mannitol. In certain other embodiments, the aqueous ophthalmic solution comprises 4% (w/v) mannitol. In certain embodiments, the alkali metal acetate is sodium acetate. In certain other embodiments, the aqueous ophthalmic solution comprises 3 mM sodium acetate. In still other embodiments, the aqueous ophthalmic solution consists of (i) about 0.5% (w/v) to about 2% (w/v) of phentolamine mesylate; (ii) about 1% (w/v) to about 6% (w/v) of one or more polyol compounds selected from the group consisting of mannitol, glycerol, and propylene glycol; (iii) about 1 mM to about 6 mM of an alkali metal acetate; (iv) acetic acid; and (v) water; wherein the solution has a pH in the range of 4.5 to 5.5.

Another such exemplary solution is an aqueous ophthalmic solution free of a chelating agent comprising: (a) about 0.25% (w/v) to about 2% (w/v) of phentolamine mesylate; (b) about 1% (w/v) to about 6% (w/v) of at least one polyol compound selected from the group consisting of is mannitol, glycerol, and propylene glycol; (c) about 1 mM to about 6 mM of an alkali metal acetate; and (d) water; wherein the solution has a pH in the range of 4.5 to 5.5 and does not contain a chelating agent.

The aqueous ophthalmic solution may be more specifically defined according to the following embodiments. For example, in certain embodiments, the aqueous ophthalmic solution comprises from about 0.25% (w/v) to about 1% (w/v) of phentolamine mesylate. In certain other embodiments, the aqueous ophthalmic solution comprises from about 1% (w/v) to about 4% (w/v) mannitol. In certain other embodiments, the aqueous ophthalmic solution comprises 4% (w/v) mannitol. In certain embodiments, the alkali metal acetate is sodium acetate. In certain other embodiments, the aqueous ophthalmic solution comprises 3 mM sodium acetate. In still other embodiments, the aqueous ophthalmic solution consists of (i) about 0.5% (w/v) to about 1% (w/v) of phentolamine mesylate; (ii) about 1% (w/v) to about 6% (w/v) of one or more polyol compounds selected from the group consisting of mannitol, glycerol, and propylene glycol; (iii) about 1 mM to about 6 mM of an alkali metal acetate; (iv) acetic acid; and (v) water; wherein the solution has a pH in the range of 4.5 to 5.5.

Further exemplary aqueous ophthalmic solutions are provided in Tables 1-3 below, where in each instance the solution has a pH in the range of 4.7 to 5.1.

TABLE 1
EXEMPLARY AQUEOUS OPHTHALMIC SOLUTIONS.

| | Formulation No. | | | | | | | |
|---|---|---|---|---|---|---|---|---|
| Component | A1 | B1 | C1 | D1 | E1 | F1 | G1 | H1 |
| Phentolamine mesylate | 1.5 | 1 | 0.5 | 1 | 1 | 1 | 1 | 1 |
| (% w/v) | | | | | | | | |
| Mannitol (% w/v) | 4 | 4 | 4 | 3 | 3 | 2 | 2 | 4 |
| Sodium acetate (mM) | 3 | 3 | 3 | 3 | 3 | 3 | 3 | 3 |
| Glycerol (% w/v) | 0 | 0 | 0 | 0.5 | 0 | 1 | 0 | 0 |
| Propylene glycol | 0 | 0 | 0 | 0 | 0.5 | 0 | 1 | 0 |
| (% w/v) | | | | | | | | |
| Dextran 70 (% w/v) | 0 | 0 | 0 | 0 | 0 | 0 | 0 | 0.1 |
| Water | q.s. | q.s. | q.s. | q.s. | q.s. | q.s. | q.s. | q.s. |

TABLE 2
EXEMPLARY AQUEOUS OPHTHALMIC SOLUTIONS.

| | Formulation No. | | | | | |
|---|---|---|---|---|---|---|
| Component | A2 | B2 | C2 | D2 | E2 | F2 |
| Phentolamine mesylate | 0.25 | 0.25 | 0.25 | 0.25 | 0.25 | 0.25 |
| (% w/v) | | | | | | |
| Mannitol (% w/v) | 4 | 3 | 3 | 2 | 2 | 4 |
| Sodium acetate (mM) | 3 | 3 | 3 | 3 | 3 | 3 |
| Glycerol (% w/v) | 0 | 0.5 | 0 | 1 | 0 | 0 |
| Propylene glycol | 0 | 0 | 0.5 | 0 | 1 | 0 |
| (% w/v) | | | | | | |
| Dextran 70 (% w/v) | 0 | 0 | 0 | 0 | 0 | 0.1 |
| Water | q.s. | q.s. | q.s. | q.s. | q.s. | q.s. |

TABLE 3
EXEMPLARY AQUEOUS OPHTHALMIC SOLUTIONS.

| | Formulation No. | | | | | | | |
|---|---|---|---|---|---|---|---|---|
| Component | A3 | B3 | C3 | D3 | E3 | F3 | G3 | H3 |
| Phentolamine mesylate | 1.5 | 1 | 0.5 | 0.25 | 1 | 1 | 1 | 1 |
| (% w/v) | | | | | | | | |
| Mannitol (% w/v) | 4 | 4 | 4 | 4 | 3 | 2 | 2 | 4 |
| Sodium acetate (mM) | 3 | 3 | 3 | 3 | 3 | 3 | 2 | 3 |
| Water | q.s. | q.s. | q.s. | q.s. | q.s. | q.s. | q.s | q.s. |

Another exemplary aqueous ophthalmic solution comprises phentolamine mesylate (e.g., at 100 w/v), mannitol (e.g., at 4% w/v), dextran having a weight average molecular weight of about 70,000 g/mol (e.g., at 0.1% w/v), hydroxypropyl methylcellulose (e.g., at 0.3% w/v), potassium chloride, purified water, sodium borate, and sodium chloride; wherein the solution has a pH in the range of about 4 to about 6. In certain embodiments, the solution has a pH in the range of 4.5 to 5.1. In certain embodiments, the aqueous ophthalmic solution consists essentially of phentolamine mesylate (e.g., at 1% w/v), mannitol (e.g., at 4% w/v), dextran having a weight average molecular weight of about 70,000 g/mol (e.g., at 0.1% w/v), hydroxypropyl methylcellulose (e.g., at 0.3% w/v), potassium chloride, purified water, sodium borate, and sodium chloride; wherein the solution has a pH in the range of 4 to 6. In certain other embodiments, the aqueous ophthalmic solution consists of phentolamine mesylate (e.g., at 1% w/v), mannitol (e.g., at 4% w/v), dextran having a weight average molecular weight of about 70,000 g/mol (e.g., at 0.1% w/v), hydroxypropyl methylcellulose (e.g., at 0.3% w/v), potassium chloride, purified water, sodium borate, and sodium chloride; wherein the solution has a pH in the range of 4.5 to 5.1.

Another exemplary aqueous ophthalmic solution comprises phentolamine mesylate (e.g., at 1% w/v), mannitol (e.g., at 4% w/v), sodium acetate (e.g., at 3 mM), and water, wherein the solution has a pH in the range of about 4 to about 6. In certain embodiments, the solution has a pH in the range of 4.5 to 5.1. In certain embodiments, the aqueous ophthalmic solution consists essentially of phentolamine mesylate (e.g., at 1% w/v), mannitol (e.g., at 4% w/v), sodium acetate (e.g., at 3 mM), and water, wherein the solution has a pH in the range of 4 to 6. In certain embodiments, the aqueous ophthalmic solution comprises phentolamine mesylate at 1% w/v, mannitol 4% w/v, sodium acetate at 3 mM, and water, wherein the solution has a pH in the range of 4.5 to 5.1. In certain other embodiments, the aqueous ophthalmic solution consists of phentolamine mesylate (e.g., at 1% w/v), mannitol (e.g., at 4% w/v), sodium acetate (e.g., at 3 mM), and water, wherein the solution has a pH in the range of 4.5 to 5.1. In certain embodiments, the aqueous ophthalmic solution consists essentially of phentolamine mesylate at 1% w/v, mannitol 4% w/v, sodium acetate at 3 mM, and water, wherein the solution has a pH in the range of 4.5 to 5.1.

Yet another exemplary solution is an aqueous ophthalmic solution free of a chelating agent that comprises: (a) about 0.1% (w/v) to about 2% (w/v) of phentolamine mesylate; (b) about 1% (w/v) to about 6% (w/v) of at least one polyol compound selected from the group consisting of is mannitol, glycerol, and propylene glycol; (c) about 1 mM to about 6 mM of an alkali metal acetate; and (d) water; wherein the solution has a pH in the range of 4 to 6 and does not contain a chelating agent.

Yet another exemplary solution is an aqueous ophthalmic solution free of a chelating agent that comprises: (a) about 0.25% (w/v) to about 2% (w/v) of phentolamine mesylate; (b) about 3% (w/v) to about 5% (w/v) of mannitol; (c) about 2 mM to about 4 mM of sodium acetate; and (d) water; wherein the solution has a pH in the range of 4.6 to 5.2 and does not contain a chelating agent.

Yet another exemplary solution is an aqueous ophthalmic solution free of a chelating agent that comprises: (a) about 0.1% (w/v) to about 2% (w/v) of phentolamine mesylate; (b) about 3% (w/v) to about 5% (w/v) of mannitol; (c) about 2 mM to about 4 mM of sodium acetate; and (d) water; wherein the solution has a pH in the range of 4.6 to 5.2 and does not contain a chelating agent. In certain embodiments, the aqueous ophthalmic solution free of a chelating agent that comprises about 0.25% (w/v) to about 1% (w/v) of phentolamine mesylate.

Yet another exemplary solution is an aqueous ophthalmic solution free of a chelating agent, comprising: (a) about 0.25% (w/v) to about 2% (w/v) of phentolamine mesylate; (b) about 3% (w/v) to about 5% (w/v) of mannitol; (c) about 2 mM to about 4 mM of sodium acetate; and (d) water; wherein the solution has a pH in the range of 4.5 to 5.2 and does not contain a chelating agent.

Yet another exemplary solution is an aqueous ophthalmic solution free of a chelating agent that comprises: (a) about 0.5% (w/v) to about 2% (w/v) of phentolamine mesylate; (b) about 3% (w/v) to about 5% (w/v) of mannitol; (c) about 2 mM to about 4 mM of sodium acetate; and (d) water; wherein the solution has a pH in the range of 4.6 to 5.2 and does not contain a chelating agent.

Yet another exemplary solution is an aqueous ophthalmic solution free of a chelating agent that comprises: (a) about 0.5% (w/v) to about 1% (w/v) of phentolamine mesylate; (b) about 3% (w/v) to about 5% (w/v) of mannitol; (c) about 1 mM to about 4 mM of sodium acetate; and (d) water; wherein the solution has a pH in the range of 4.6 to 5.2 and does not contain a chelating agent.

Yet another exemplary solution is an aqueous ophthalmic solution free of a chelating agent, comprising: (a) about 0.1% (w/v) to about 1% (w/v) of phentolamine mesylate; (b) about 4% mannitol; (c) about 3 mM sodium acetate; and (d) water; wherein the solution has a pH in the range of 4.6 to 5.2 and does not contain a chelating agent. In certain embodiments, the aqueous ophthalmic solution free of a chelating agent that comprises about 0.25% (w/v) to about 1% (w/v) of phentolamine mesylate.

Yet another exemplary solution is an aqueous ophthalmic solution free of a chelating agent, comprising: (a) about 0.5% (w/v) to about 1% (w/v) of phentolamine mesylate; (b) about 4% mannitol; (c) about 3 mM sodium acetate; and (d) water; wherein the solution has a pH in the range of 4.6 to 5.2 and does not contain a chelating agent.

Yet another exemplary solution is an aqueous ophthalmic solution free of a chelating agent, comprising: (a) about 0.25% (w/v) to about 2% (w/v) of phentolamine mesylate; (b) about 3% (w/v) to about 5% (w/v) of mannitol; (c) about 2 mM to about 4 mM of a buffer comprising sodium acetate; and (d) water; wherein the solution has a pH in the range of 4.5 to 5.2 and does not contain a chelating agent.

Yet another exemplary solution is an aqueous ophthalmic solution free of a chelating agent that comprises: (a) about 0.5% (w/v) to about 2% (w/v) of phentolamine mesylate; (b) about 3% (w/v) to about 5% (w/v) of mannitol; (c) about 2 mM to about 4 mM of a buffer comprising sodium acetate; and (d) water; wherein the solution has a pH in the range of 4.6 to 5.2 and does not contain a chelating agent.

Yet another exemplary solution is an aqueous ophthalmic solution free of a chelating agent that comprises: (a) about 0.5% (w/v) to about 1% (w/v) of phentolamine mesylate; (b) about 3% (w/v) to about 5% (w/v) of mannitol; (c) about 1 mM to about 4 mM of a buffer comprising sodium acetate; and (d) water; wherein the solution has a pH in the range of 4.6 to 5.2 and does not contain a chelating agent.

Yet another exemplary solution is an aqueous ophthalmic solution free of a chelating agent, comprising: (a) about 0.1% (w/v) to about 1% (w/v) of phentolamine mesylate; (b) about 4% mannitol; (c) about 3 mM of a buffer comprising sodium acetate; and (d) water; wherein the solution has a pH in the range of 4.6 to 5.2 and does not contain a chelating agent. In certain embodiments, the aqueous ophthalmic solution free of a chelating agent that comprises about 0.25% (w/v) to about 1% (w/v) of phentolamine mesylate.

Yet another exemplary solution is an aqueous ophthalmic solution free of a chelating agent, comprising: (a) about 0.5% (w/v) to about 1% (w/v) of phentolamine mesylate; (b) about 4% mannitol; (c) about 3 mM of a buffer comprising sodium acetate; and (d) water; wherein the solution has a pH in the range of 4.6 to 5.2 and does not contain a chelating agent.

### Stability Features of Aqueous Ophthalmic Solutions

The aqueous ophthalmic solutions described herein may be further characterized according to their stability features, such as the percentage of phentolamine (or pharmaceutically acceptable salt thereof) that is present in the aqueous ophthalmic solution after storage for a certain length of time. As explained above, one of the benefits of the present aqueous ophthalmic solutions is that they possess good stability over extended periods of time, even though they do not have a chelating agent.

Accordingly, in certain embodiments, the aqueous ophthalmic solution is characterized by less than 2% by weight of the phentolamine or pharmaceutically acceptable salt thereof degrades upon storage of the solution at 25° C. for 12 weeks. In certain other embodiments, the aqueous ophthalmic solution is characterized by less than 2% by weight of the phentolamine or pharmaceutically acceptable salt thereof degrades upon storage at 25° C. for 24 weeks (or 36 weeks or 48 weeks). In yet other embodiments, less than 7% by weight of the phentolamine or pharmaceutically acceptable salt thereof degrades upon storage at 40° C. for 12 weeks (or 24, 36, or 48 weeks). In yet other embodiments, the aqueous ophthalmic solution is characterized by less than 10% by weight of the phentolamine or pharmaceutically acceptable salt thereof degrades upon storage at 25° C. for 18 months, 24 months, or 36 months. In yet other embodiments, the aqueous ophthalmic solution is characterized by less than 10% by weight of the phentolamine or pharmaceutically acceptable salt thereof degrades upon storage at temperature in the range of 2-8° C. for 18 months, 24 months, or 36 months. In yet other embodiments, the aqueous ophthalmic solution is characterized by less than 4% by weight (or preferably less than 3% by weight) of the phentolamine or pharmaceutically acceptable salt thereof degrades upon storage at 25° C. for 18 months, 24 months, or 36 months. In yet other embodiments, less than 10% by weight of the phentolamine or pharmaceutically acceptable salt thereof degrades upon storage at 40° C. for 4, 5, or 6 months.

### Polyvinylpyrrolidone Artificial Tears Formulation

Another ophthalmic solution contemplated for use in the present invention is an ophthalmic solution comprising an alpha-adrenergic antagonist (e.g., phentolamine or a pharmaceutically acceptable salt thereof) and a polyvinylpyrrolidone artificial tears composition. Exemplary polyvinylpyrrolidone artificial tears compositions are described in, for example, U.S. Pat. Nos. 5,895,654; 5,627,611; and 5,591,426; and U.S. Patent Application Publication No. 2002/0082288, all of which are hereby incorporated by reference. Artificial tears compositions are understood to promote wettability and spread, have good retention and stability on the eye, and desirably do not cause any significant discomfort to the user. Accordingly, an exemplary polyvinylpyrrolidone artificial tear composition comprises: (1) polyvinylpyrrolidone, preferably in the amount of about 0.1-5% by weight of the solution; (2) benzalkonium chloride, preferably in an amount of about 0.01-0.10% by weight of the solution; (3) hydroxypropyl methylcellulose, preferably in an amount of about 0.2-1.5% by weight of the solution; (4) glycerin, preferably in an amount of about 0.2-1.0% by weight of the solution, and (5) water, wherein the composition is an aqueous solution having isotonic properties.

### Sustained Release Delivery Systems

When it is desirable to have sustained release of one or more therapeutic agents to the patient, the therapeutic agent(s) may be administered to the patient in the form of a sustained release delivery system. Sustained release delivery systems are described in the published literature. Exemplary sustained release delivery systems include intracanalicular inserts, a slow release contact lens, a bio-erodible IVT insert, and an intracameral insert. Inserts may be biodegradable or non-biodegradable. Exemplary materials described in the literature for use in sustained release delivery systems include polyethylene glycol, a mixture of EVA and PVA polymers, a mixture of silicone and PVA polymer, a mixture of polyimide and PVA polymer, a mixture of PMMA and EVA polymers, PLGA polymer, liposomes, and silicon oxide (e.g., silicon oxide particles, silicon oxide beads, and silicon oxide porous nanoparticles). Additional exemplary sustained release delivery systems are silicon oxide sustained release delivery systems that are commercially available, such as from Duxeltech.

### V. Medical Kits

Another aspect of the invention provides a medical kit comprising, for example, (i) a therapeutic agent described herein, and (ii) instructions for treating mydriasis, glaucoma, and other ocular conditions according to methods described herein.

### EXAMPLES

The invention now being generally described, will be more readily understood by reference to the following examples, which are included merely for purposes of illustrating certain aspects and embodiments of the present invention, and are not intended to limit the invention.

### Example 1—Reversal of Mydriasis in Human Subjects Using Phentolamine Mesylate in Combination with Pilocarpine Hydrochloride

Ability of phentolamine mesylate in combination with pilocarpine hydrochloride to reverse pharmacologically induced mydriasis in the eye of a human subject may be evaluated according to a clinical study. Exemplary procedures are described below.

At least 20 subjects are enrolled and randomized 1:1 into one of two treatment sequences. All subjects are first administered a mydriatic agent (phenylephrine (2.5% w/w) or tropicamide (1% w/w)) by delivery of an eye drop containing the mydriatic agent to the subject's eyes. Then, approximately one hour after receiving the mydriatic agent, the subject is administered study medication according to Treatment Protocol 1 or Treatment Protocol 2.

In Treatment Protocol 1, the subject receives placebo on the first treatment day (Visit 1/Day 1) and receives one eye drop of 1% w/w Phentolamine Mesylate Ophthalmic Solution along with one eye drop of 0.4% w/w Pilocarpine Hydrochloride Ophthalmic Solution on the second treatment day (Visit 2/Day 8+2 days). In Treatment Protocol 2, the subject receives one eye drop of 1% w/w Phentolamine Mesylate Ophthalmic Solution along with one eye drop of 0.4% w/w Pilocarpine Hydrochloride Ophthalmic Solution on the first treatment day (Visit 1/Day 1) and receives placebo on the second treatment day (Visit 2/Day 8+2 days). The study eye is defined as the eye with the larger pupil diameter at maximum (1 hour after instillation of the mydriatic agent) at Visit 1. If both eyes have the same pupil diameter at maximum, then the study eye is the right eye.

All treatments are administered to both eyes in the subject. At each visit, pupil diameter, accommodation, near and distance visual acuity (VA) and redness in each eye is measured before (−1 hour/baseline) and 1 hour after (0 minutes/maximum) the mydriatic agent instillation in each eye (i.e., right before the study treatment is administered), and at 30 minutes, 1 hour, 2 hours, 4 hours and 6 hours after treatment dosing. Efficacy and safety are evaluated, which includes analysis of reduction in pupil diameter.

TABLE 1
Study Medication

| Study Medication | Composition of Study Medication |
|---|---|
| 1% w/w Phentolamine | 1% w/w phentolamine mesylate |
| Mesylate Ophthalmic | 4% w/w mannitol |
| Solution | 3 mM buffer comprising sodium acetate and acetic |
| | acid |
| | water |
| | pH in the range 4.8 to 5.0 |
| 0.4% w/w Pilocarpine | 0.4% w/w pilocarpine hydrochloride |
| Hydrochloride | water |
| Ophthalmic Solution | |
| Placebo | 4% w/w mannitol |
| | 3 mM buffer comprising sodium acetate and acetic |
| | acid |
| | water |
| | pH in the range 4.8 to 5.0 |

### Analysis of Efficacy

A primary efficacy endpoint is the change in pharmacologically induced mydriatic (max) pupil diameter (0 minutes) at 2 hours post-treatment in the study eye. The study eye is defined as the eye with the larger pupil diameter at maximum (1 hour after instillation of the mydriatic agent) at Visit 1. If both eyes have the same pupil diameter at maximum, then the study eye is the right eye. This is the study eye for both Visit 1 and Visit 2 assessments.

Secondary efficacy endpoints (for the study eye; for the non-study eye; and for both eyes) include:
Change (in mm) from max pupil diameter (0 minutes) at each remaining timepoint (30 min, 1 hour, 4 hours, 6 hours).
Percentage of subjects who achieved a pre-specified reduction of ≥2 mm, ≥3 mm, and ≥4 mm from max pupil diameter at each timepoint (30 min, 1 hour, 2 hours, 4 hours, 6 hours).
Percentage of subjects who achieved pupil diameter of no more than 0.5 mm above baseline (−1 hour) at each timepoint (0 min, 30 min, 1 hour, 2 hours, 4 hours, 6 hours).
Change from baseline (−1 hour) in accommodation at each timepoint (0 min, 30 min, 1 hour, 2 hours, 4 hours, 6 hours).
Percent of subjects with unchanged accommodation from baseline (−1 hour) at each timepoint (0 min, 30 min, 1 hour, 2 hours, 4 hours, 6 hours).
Change from baseline (−1 hour) in BCDVA at each timepoint (0 min, 30 min, 1 hour, 2 hours, 4 hours, 6 hours).
Percent of subjects who returned to baseline (−1 hour) BCDVA at each timepoint (0 min, 30 min, 1 hour, 2 hours, 4 hours, 6 hours).
Change from baseline (−1 hour) in DCNVA at each timepoint (0 min, 30 min, 1 hour, 2 hours, 4 hours, 6 hours).
Percent of subjects who returned to baseline (−1 hour) DCNVA at each timepoint (0 min, 30 min, 1 hour, 2 hours, 4 hours, 6 hours).

### Example 2—Treating Acute Angle-Closure Glaucoma Using Phentolamine Mesylate in Human Subjects

Ability of phentolamine mesylate to treat acute angle-closure glaucoma in human subjects may be evaluated according to a clinical study. Exemplary procedures are described below.

At least 20 human subjects with acute angle-closure glaucoma are randomized. Subjects are randomized in a 1:1 ratio to receive one eye drop of 1% w/w Phentolamine Mesylate Ophthalmic Solution or placebo. Efficacy evaluations include measurement of intraocular pressure (IOP).

TABLE 2
Study Medication

| Study Medication | Composition of Study Medication |
|---|---|
| 1% w/w Phentolamine | 1% w/w phentolamine mesylate |
| Mesylate Ophthalmic | 4% w/w mannitol |
| Solution | 3 mM buffer comprising sodium acetate and acetic |
| | acid |
| | water |
| | pH in the range 4.8 to 5.0 |
| Placebo Ophthalmic | 4% w/w mannitol |
| Solution | 3 mM buffer comprising sodium acetate and acetic |
| | acid |
| | water |
| | pH in the range 4.8 to 5.0 |

### Example 3—Reducing Pupil Diameter and Improving Visual Performance in Human Subjects Using Phentolamine Mesylate in Combination with Pilocarpine Hydrochloride

Ability of phentolamine mesylate in combination with pilocarpine hydrochloride to reduce pupil diameter and improve visual performance in human subjects was evaluated according to a randomized, double-masked, placebo-controlled clinical study. Approximately 152 subjects were enrolled and randomized into one of four treatment groups. Experimental procedures and results are provided below.

### Part I—Experimental Procedures

Human subjects were screened for potential enrollment and, if qualified, enrolled in the study. Inclusion criteria and exclusion criteria used to select subjects for enrollment in the study are provided below. Following the enrollment period, approximately 152 subjects were enrolled and randomized into one of four treatment groups. The four treatment groups were:
Group I—1% w/w Phentolamine Mesylate Ophthalmic Solution and 0.4% w/w Pilocarpine Hydrochloride Ophthalmic Solution
Group II—1% w/w Phentolamine Mesylate Ophthalmic Solution
Group III—Placebo Ophthalmic Solution and 0.4% w/w Pilocarpine Hydrochloride Ophthalmic Solution
Group IV—Placebo Ophthalmic Solution.

During the Screening/Baseline visit (i.e., Visit 1), the subject was evaluated for distance-corrected near visual acuity (under both mesopic and photopic conditions), best-corrected distance visual acuity (under both mesopic and photopic conditions), best-corrected intermediate visual acuity (under photopic conditions), pupil diameter, biomicroscopy, intraocular pressure measurement, and visual assessment of conjunctival hyperemia. Also during Visit 1, the subject was provided the 1% w/w Phentolamine Mesylate Ophthalmic Solution or the Placebo Ophthalmic Solution and instructed to topically administer the foregoing, according to treatment group, to their eye at or near the bedtime for 3 to 4 consecutive days immediately prior to Visit 2.

At Visit 2, one eye drop of 0.4% w/w Pilocarpine Hydrochloride Ophthalmic Solution was administered to the subject's eye for subjects in treatment groups I and III. All subjects were determined to have presbyopia, and all treatments were administered to both eyes of the subject. At Visit 2, pupil diameter, distance-corrected near visual acuity (under both mesopic and photopic conditions), best-corrected distance visual acuity (under both mesopic and photopic conditions), and best-corrected intermediate visual acuity (under photopic conditions) were measured prior to administration of any 0.4% w/w Pilocarpine Hydrochloride Ophthalmic Solution. After administration of any 0.4% w/w Pilocarpine Hydrochloride Ophthalmic Solution during Visit 2, subjects were be assessed at multiple time points from 30 minutes to 6 hours for the following: pupil diameter, distance-corrected near visual acuity (under both mesopic and photopic conditions), best-corrected distance visual acuity (under both mesopic and photopic conditions), and best-corrected intermediate visual acuity (under photopic conditions), conjunctival hyperemia, adverse events, and subject ocular tolerability. Biomicroscopy, measurement of intraocular pressure, and ophthalmoscopy were performed at 6 hours after administration of any 0.4% w/w Pilocarpine Hydrochloride Ophthalmic Solution during Visit 2. At 1-3 days after Visit 2, subjects participated in a follow-up call to assess adverse events.

When taking measurements:
DCNVA Reading/Near were measured under photopic and mesopic conditions by a high-contrast Near Visual Acuity Chart in the Precision Vision Small 914 Illuminator Cabinet (light box) at 16 inches (˜40 cm).
BCDVA was measured under photopic conditions by a high-contrast Standard Early Treatment Diabetic Retinopathy Study (ETDRS) illuminated chart (on wall or stand) at 4 m.
BCIVA was measured under photopic conditions by a high-contrast Near Visual Acuity Chart in the Precision Vision Small 914 Illuminator Cabinet (light box) at 26 inches (66 cm).
Pupil diameter (PD) was measured with a NeurOptics pupillometer (mm)
Conjunctival hyperemia was measured with CCLRU images using a 4-point scale (0-3).
   None (0)=Normal. Appears white with a small number of conjunctival blood vessels easily observed
   Mild (+1)=Prominent, pinkish-red color of both the bulbar and palpebral conjunctiva
   Moderate (+2)=Bright, scarlet red color of the bulbar and palpebral conjunctiva
   Severe (+3)=Beefy red with petechiae, dark red bulbar and palpebral conjunctiva with evidence of subconjunctival hemorrhage.

In photopic lighting conditions, the distance and near illuminated charts will be at a luminance level of approximately 85 to 160 cd/m² (85 to 160 nits). In mesopic conditions, the distance and near illuminated charts will be at a luminance level of approximately 3 cd/m² (3 nits). Ambient lighting in both photopic and mesopic is only the luminance level of the light box. The luminance reduction for mesopic conditions is done by way of a neutral density filter that reduces the luminance. Subjects will be allowed to acclimate to these lighting conditions (with the eyes open normally for a minimum of 2 minutes prior to the set of PD and visual acuity (VA) measurements). Subjects were to sit in the exam chair facing directly at the illuminated chart during the acclimation period and for all assessments.

Room lights were to be on for the scheduled remaining safety assessments (e.g., conjunctival hyperemia, AEs, subject questionnaire, etc.). The subject is to be in the same room for all assessments, and every effort was be made to have the same person perform the measurements at all visits.

For visual acuity, measurements were made in letters and converted to LogMAR and lines, as appropriate.

### Inclusion Criteria

1. Males or females ≥40 and ≤64 years of age.
2. Able to comply with all protocol-mandated procedures independently and to attend all scheduled office visits.
3. Able and willing to give signed informed consent.
4. Able to self-administer study medication throughout the study period.
5. Best-corrected distance visual acuity (BCDVA) of 0.0 LogMAR (20/20 Snellen equivalent) or better in each eye under photopic conditions.
6. Distance-corrected near visual acuity (DCNVA) of 0.4 LogMAR (20/50 Snellen equivalent) or worse under photopic conditions in each eye and binocularly.
7. Subjects who depend on reading glasses or bifocals in which their binocular best-corrected near visual acuity is 0.1 LogMAR (20/25 Snellen equivalent) or better.

### Exclusion Criteria

### Ophthalmic Conditions (In Either Eye):

1. Use of any topical prescription or over-the-counter (OTC) ophthalmic medications of any kind within 7 days of Screening until study completion, with the exception of lid scrubs with OTC products (e.g., OCuSOFT® lid scrub, SteriLid®, baby shampoo, etc.).
2. Use of any over-the-counter (OTC) artificial tears (preserved or unpreserved) at least once per day within 7 days of Screening until study completion.
3. Current use of any topical ophthalmic therapy for dry eye (e.g., Restasis, Xiidra, etc.).
4. Tear break-up time of <5 seconds or corneal fluorescein staining (≥grade 2 in the inferior zone or ≥grade 1 in the central zone using the National Eye Institute scale).
5. Clinically significant ocular disease (e.g., cataract, glaucoma, corneal edema, uveitis, retinal degeneration, loss of visual field, any macular pathology) that might interfere with the study as deemed by the investigator.
6. Recent or current evidence of ocular infection or inflammation in either eye (such as current evidence of clinically significant blepharitis, conjunctivitis, keratitis, etc.). Subjects must be symptom free for at least 7 days.
7. Any history of herpes simplex or herpes zoster keratitis.
8. History of diabetic retinopathy or diabetic macular edema.
9. Known allergy, hypersensitivity, or contraindication to any component of the phentolamine, pilocarpine, or vehicle formulations.
10. History of cauterization of the punctum or punctal plug (silicone or collagen) insertion or removal.
11. Ocular trauma, ocular surgery (e.g., intraocular lenses), ocular laser treatment within the 6 months prior to Screening. Any subject with multifocal intraocular lenses are excluded.
12. History of any traumatic (surgical or nonsurgical) or non-traumatic condition affecting the pupil or iris (e.g., irregularly shaped pupil, neurogenic pupil disorder, iris atrophy, iridotomy, iridectomy, iritis, etc.).
13. Unwilling or unable to discontinue use of contact lenses at Screening until study completion.
14. Conjunctival hyperemia ≥grade 2 on the Cornea and Contact Lens Research Unit (CCLRU) 4-point scale.

### Systemic Conditions

15. Known hypersensitivity or contraindication to alpha- and/or beta-adrenoceptor antagonists (e.g., chronic obstructive pulmonary disease or bronchial asthma; abnormally low blood pressure (BP) or heart rate (HR); second- or third-degree heart blockage or congestive heart failure).
16. Known hypersensitivity or contraindication to any systemic cholinergic parasympathomimetic agents.
17. Clinically significant systemic disease (e.g., uncontrolled diabetes, myasthenia gravis, cancer, hepatic, renal, endocrine, or cardiovascular disorders) that might interfere with the study as deemed by the Investigator.
18. Initiation of treatment with, or any changes to, the current dosage, drug, or regimen of any systemic adrenergic or cholinergic drugs within 7 days prior to Screening or during the study.
19. Participation in any investigational study within 30 days prior to Screening.
20. Females of childbearing potential who are pregnant, nursing, planning a pregnancy, or not using a medically acceptable form of birth control. Acceptable methods include the use of at least one of the following: intrauterine device (IUD), hormonal (oral, injection, patch, implant, ring), barrier with spermicide (condom, diaphragm), or abstinence. A female is considered to be of childbearing potential, unless she is 1 year postmenopausal or 3 months post-surgical sterilization. All females of childbearing potential including those with post-tubal ligation must have a negative urine pregnancy test result at Visit 1 (Screening/Baseline).
21. Resting HR outside the specified range of 50 to 110 beats per minute following at least a 5-minute rest period in the sitting position at the Screening Visit 1. HR may be repeated only once if outside the specified range, following another 5-minute rest period in the sitting position.
22. Hypertension with resting diastolic BP>105 mmHg or systolic BP>160 mmHg following at least a 5-minute rest period in the sitting position at the Screening Visit 1. BP may be repeated only once if outside the specified range, following another 5-minute rest period in the sitting position.

Study Medication used in the clinical study is as set forth in the following table.

TABLE 1
Study Medication

| Study Medication | Composition of Study Medication |
|---|---|
| 1% w/w Phentolamine | 1% w/w phentolamine mesylate |
| Mesylate Ophthalmic | 4% w/w mannitol |
| Solution | 3 mM buffer comprising sodium acetate and acetic |
| | acid |
| | water |
| | pH in the range 4.8 to 5.0 |
| Placebo Ophthalmic | 4% w/w mannitol |
| Solution | 3 mM buffer comprising sodium acetate and acetic |
| | acid |
| | water |
| | pH in the range 4.8 to 5.0 |
| 0.4% w/w Pilocarpine | 0.4% w/w pilocarpine hydrochloride |
| Hydrochloride | 0.01% benzalkonium chloride |
| Ophthalmic Solution | 0.5% hypromellose 2910 |
| | boric acid |
| | sodium chloride |
| | sodium citrate |
| | water |
| | pH in the range 3.5 to 5.5 |

### Part II—Results

Data on observed reduction in pupil diameter is provided in Table 2 below. Data on observed improvement in best-corrected distance visual acuity (BCDVA) is provided in Table 3 below. Data on observed intraocular pressure is provided in Table 4 below.

TABLE 2
Change in Mesopic Pupil Diameter (PD) by Time Point

| | Pupil Diameter (mm) | |
|---|---|---|
| | | 1% w/w Phentolamine Mesylate Ophthalmic |
| | | Solution + 0.4% w/w Pilocarpine Hydrochloride |
| Time | Placebo | Ophthalmic Solution |
| (hr) | (n = 43) | (n = 43) |
| Baseline | 5.13 | 5.06 |
| 0 | 5.15 | 3.8 |
| 0.5 | 5.12 | 2.31 |
| 1 | 5.21 | 2.16 |
| 2 | 5.13 | 2.43 |
| 3 | 5.11 | 2.75 |
| 4 | 5.13 | 2.99 |
| 6 | 5 | 3.24 |

TABLE 3
Change in BCDVA by Time Point

| | BCDVA | |
|---|---|---|
| | | 1% w/w Phentolamine Mesylate Ophthalmic |
| | | Solution + 0.4% w/w Pilocarpine |
| | Placebo | Hydrochloride Ophthalmic Solution |
| Time (hr) | (n = 43) | (n = 43) |
| Baseline | 0 | 0 |
| 0 | 0.47 | 1.41 |
| 1 | 1.18 | 1.8 |
| 3 | 1.08 | 1.39 |
| 6 | 0.43 | 0.95 |

TABLE 4
Intraocular Pressure by Time Point

| | Intraocular Pressure (mmHg) | |
|---|---|---|
| | | 1% w/w Phentolamine Mesylate Ophthalmic |
| | | Solution + 0.4% w/w Pilocarpine Hydrochloride |
| | Placebo | Ophthalmic Solution |
| Time (hr) | (n = 43) | (n = 43) |
| Baseline | 13.6 | 14.5 |
| 6 | 13.7 | 14.1 |

### INCORPORATION BY REFERENCE

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method of treating mydriasis in a patient, comprising administering to an eye of a patient in need thereof a dosage of an alpha-adrenergic antagonist and a dosage of a muscarinic acetylcholine receptor agonist in an amount effective to thereby treat the mydriasis.

2. The method of claim 1, wherein the patient suffers from glaucoma.

3. A method of treating mydriasis in a patient suffering from glaucoma while reducing the risk of an angle-closure glaucoma attack, comprising administering to an eye of a patient in need thereof a dosage of an alpha-adrenergic antagonist in an amount effective to thereby treat the mydriasis and reduce the risk of an angle-closure glaucoma attack.

4. The method of claim 3, further comprising administering to the eye of the patient in need thereof a muscarinic acetylcholine receptor agonist.

5. The method of any one of claims 1, 2, or 4, wherein the muscarinic acetylcholine receptor agonist is pilocarpine or a pharmaceutically acceptable salt thereof.

6. The method of any one of claims 1, 2, or 4, wherein the muscarinic acetylcholine receptor agonist is a pharmaceutically acceptable salt of pilocarpine.

7. The method of any one of claims 1, 2, or 4, wherein the muscarinic acetylcholine receptor agonist is pilocarpine hydrochloride.

8. The method of any one of claims 1, 2, or 4-7, wherein the dosage of muscarinic acetylcholine receptor agonist is less than about 2 mg.

9. The method of any one of claims 1, 2, or 4-7, wherein the dosage of muscarinic acetylcholine receptor agonist is less than about 1 mg.

10. The method of any one of claims 1, 2, or 4-7, wherein the dosage of muscarinic acetylcholine receptor agonist is less than about 0.5 mg.

11. The method of any one of claims 1, 2, or 4-7, wherein the dosage of muscarinic acetylcholine receptor agonist is less than about 0.25 mg.

12. The method of any one of claims 1, 2, or 4-7, wherein the dosage of muscarinic acetylcholine receptor agonist is from about 0.1 mg to about 1.5 mg.

13. The method of any one of claims 1, 2, or 4-7, wherein the dosage of muscarinic acetylcholine receptor agonist is from about 0.1 mg to about 1.0 mg.

14. The method of any one of claims 1, 2, or 4-7, wherein the dosage of muscarinic acetylcholine receptor agonist is from about 0.1 mg to about 0.5 mg.

15. The method of any one of claims 1, 2, or 4-7, wherein the dosage of muscarinic acetylcholine receptor agonist is from about 0.1 mg to about 0.3 mg.

16. The method of any one of claims 1, 2, or 4-7, wherein the dosage of muscarinic acetylcholine receptor agonist is from about 0.1 mg to about 0.2 mg.

17. The method of any one of claims 1, 2, or 4-7, wherein the dosage of muscarinic acetylcholine receptor agonist is from about 0.1 mg to about 0.15 mg.

18. The method of any one of claims 1, 2, or 4-7, wherein the dosage of muscarinic acetylcholine receptor agonist is from about 0.2 mg to about 0.4 mg.

19. The method of any one of claims 1, 2, or 4-7, wherein the dosage of muscarinic acetylcholine receptor agonist is from about 0.3 mg to about 0.7 mg.

20. The method of any one of claims 1, 2, or 4-7, wherein the dosage of muscarinic acetylcholine receptor agonist is about 0.2 mg, about 0.25 mg, about 0.3 mg, about 0.35 mg, or about 0.4 mg.

21. The method of any one of claims 1, 2, or 4-20, wherein the dosage of muscarinic acetylcholine receptor agonist is administered in the form of an ophthalmic solution containing muscarinic acetylcholine receptor agonist, a lipid, and a pharmaceutically acceptable carrier.

22. The method of claim 21, wherein the muscarinic acetylcholine receptor agonist is present in the ophthalmic solution at a concentration ranging from about 0.1% (w/v) to about 4% (w/v).

23. The method of claim 21, wherein the muscarinic acetylcholine receptor agonist is present in the ophthalmic solution at a concentration ranging from about 0.2% (w/v) to about 1% (w/v).

24. The method of claim 21, wherein the muscarinic acetylcholine receptor agonist is present in the ophthalmic solution at a concentration of about 0.4% (w/v).

25. The method of claim 21, wherein the muscarinic acetylcholine receptor agonist is present in the ophthalmic solution at a concentration ranging from about 1% (w/v) to about 1.5% (w/v).

26. The method of claim 21, wherein the muscarinic acetylcholine receptor agonist is present in the ophthalmic solution at a concentration of about 1.25% (w/v).

27. The method of any one of claims 1, 2, or 4-26, wherein the dosage of muscarinic acetylcholine receptor agonist is topically administered to the eye in the form of an eye drop.

28. The method of any one of claims 1, 2, or 4-27, wherein the dosage of muscarinic acetylcholine receptor agonist is administered to the eye no more than once per day.

29. The method of any one of claims 1-28, wherein the patient suffers from glaucoma and has a narrow angle.

30. The method of any one of claims 1-29, wherein the mydriasis is pharmacologically induced mydriasis.

31. The method of any one of claims 1-29, wherein the mydriasis is due to the patient having received one or more of atropine, cyclopentolate, homatropine, scopolamine, tropicamide, flubiprofen, suprofen, hydroxyamphetamine, phenylephrine, ketorolac, or a pharmaceutically acceptable salt thereof.

32. The method of any one of claims 1-29, wherein the mydriasis is due to the patient having received one or more of atropine, cyclopentolate, homatropine, scopolamine, tropicamide, or a pharmaceutically acceptable salt thereof.

33. The method of any one of claims 1-29, wherein the mydriasis is due to the patient having received one or more of tropicamide, phenylephrine, or a pharmaceutically acceptable salt thereof.

34. A method of improving visual performance under dim light conditions in a patient, comprising administering to an eye of a patient in need thereof a dosage of an alpha-adrenergic antagonist and a dosage of a muscarinic acetylcholine receptor agonist in an amount effective to thereby improve visual performance under dim light conditions.

35. The method of claim 34, wherein the method results in an improvement in visual acuity characterized by at least a one-line improvement in the patient's vision measured using a Snellen chart.

36. The method of claim 34, wherein the method results in an improvement in visual acuity characterized by at least a two-line improvement in the patient's vision measured using a Snellen chart.

37. A method of reducing pupil diameter under dim light conditions in a patient, comprising administering to an eye of a patient in need thereof a dosage of an alpha-adrenergic antagonist and a dosage of a muscarinic acetylcholine receptor agonist in an amount effective to thereby reduce pupil diameter under dim light conditions.

38. The method of claim 37, wherein the method results in at least a 20% reduction in pupil diameter under dim light conditions.

39. The method of claim 37, wherein the method results in at least a 30% reduction in pupil diameter under dim light conditions.

40. The method of claim 37, wherein the method results in at least a 35% reduction in pupil diameter under dim light conditions.

41. A method of reducing an aberrant focus of scattered light rays in a patient's eye under dim light conditions, comprising administering to an eye of a patient in need thereof a dosage of an alpha-adrenergic antagonist and a dosage of a muscarinic acetylcholine receptor agonist in an amount effective to thereby reduce aberrant focus of scattered light rays in a patient's eye under dim light conditions.

42. The method of claim 41, wherein the method reduces aberrant focus of scattered light rays in a patient's eye under dim light conditions for at least 3 hours.

43. The method of claim 41, wherein the method reduces aberrant focus of scattered light rays in a patient's eye under dim light conditions for at least 6 hours.

44. The method of claim 41, wherein the method reduces aberrant focus of scattered light rays in a patient's eye under dim light conditions for at least 12 hours.

45. The method of claim 41, wherein the method reduces aberrant focus of scattered light rays in a patient's eye under dim light conditions for at least 24 hours.

46. The method of any one of claims 34-45, wherein the muscarinic acetylcholine receptor agonist is pilocarpine or a pharmaceutically acceptable salt thereof.

47. The method of any one of claims 34-45, wherein the muscarinic acetylcholine receptor agonist is a pharmaceutically acceptable salt of pilocarpine.

48. The method of any one of claims 34-45, wherein the muscarinic acetylcholine receptor agonist is pilocarpine hydrochloride.

49. The method of any one of claims 34-48, wherein the dosage of muscarinic acetylcholine receptor agonist is less than about 2 mg.

50. The method of any one of claims 34-48, wherein the dosage of muscarinic acetylcholine receptor agonist is less than about 1 mg.

51. The method of any one of claims 34-48, wherein the dosage of muscarinic acetylcholine receptor agonist is less than about 0.5 mg.

52. The method of any one of claims 34-48, wherein the dosage of muscarinic acetylcholine receptor agonist is less than about 0.25 mg.

53. The method of any one of claims 34-48, wherein the dosage of muscarinic acetylcholine receptor agonist is from about 0.1 mg to about 1.5 mg.

54. The method of any one of claims 34-48, wherein the dosage of muscarinic acetylcholine receptor agonist is from about 0.1 mg to about 1.0 mg.

55. The method of any one of claims 34-48, wherein the dosage of muscarinic acetylcholine receptor agonist is from about 0.1 mg to about 0.5 mg.

56. The method of any one of claims 34-48, wherein the dosage of muscarinic acetylcholine receptor agonist is from about 0.1 mg to about 0.3 mg.

57. The method of any one of claims 34-48, wherein the dosage of muscarinic acetylcholine receptor agonist is from about 0.1 mg to about 0.2 mg.

58. The method of any one of claims 34-48, wherein the dosage of muscarinic acetylcholine receptor agonist is from about 0.1 mg to about 0.15 mg.

59. The method of any one of claims 34-48, wherein the dosage of muscarinic acetylcholine receptor agonist is from about 0.2 mg to about 0.4 mg.

60. The method of any one of claims 34-48, wherein the dosage of muscarinic acetylcholine receptor agonist is from about 0.3 mg to about 0.7 mg.

61. The method of any one of claims 34-48, wherein the dosage of muscarinic acetylcholine receptor agonist is about 0.1 mg, about 0.15 mg, about 0.2 mg, about 0.25 mg, about 0.3 mg, about 0.35 mg, or about 0.4 mg.

62. The method of any one of claims 34-61, wherein the dosage of muscarinic acetylcholine receptor agonist is administered in the form of an ophthalmic solution containing muscarinic acetylcholine receptor agonist, a lipid, and a pharmaceutically acceptable carrier.

63. The method of claim 62, wherein the muscarinic acetylcholine receptor agonist is present in the ophthalmic solution at a concentration ranging from about 0.1% (w/v) to about 2% (w/v).

64. The method of claim 62, wherein the muscarinic acetylcholine receptor agonist is present in the ophthalmic solution at a concentration ranging from about 0.1% (w/v) to about 0.5% (w/v).

65. The method of claim 62, wherein the muscarinic acetylcholine receptor agonist is present in the ophthalmic solution at a concentration of about 0.4% (w/v).

66. The method of claim 62, wherein the muscarinic acetylcholine receptor agonist is present in the ophthalmic solution at a concentration ranging from about 1% (w/v) to about 1.5% (w/v).

67. The method of claim 62, wherein the muscarinic acetylcholine receptor agonist is present in the ophthalmic solution at a concentration of about 1.25% (w/v).

68. The method of any one of claims 34-67, further comprising topically administering to the eye of the patient a second dosage of a muscarinic acetylcholine receptor agonist in an amount effective to further reduce diameter of the patient's pupil.

69. The method of claim 68, wherein the second dosage of a muscarinic acetylcholine receptor agonist is an ophthalmic solution comprising muscarinic acetylcholine receptor agonist at a concentration of from about 0.1% (w/v) to about 2% (w/v).

70. The method of any one of claims 34-69, wherein the dosage of muscarinic acetylcholine receptor agonist is topically administered to the eye in the form of an eye drop.

71. The method of any one of claims 34-70, wherein the dosage of muscarinic acetylcholine receptor agonist is administered to the eye no more than once per day.

72. The method of any one of claims 34-71, wherein the method achieves a pupil diameter in the range of from about 2.5 mm to about 5.5 mm under dim light conditions.

73. The method of any one of claims 34-71, wherein the method achieves a pupil diameter in the range of from about 3 mm to about 5 mm under dim light conditions.

74. The method of any one of claims 34-71, wherein the method achieves a pupil diameter in the range of from about 3 mm to about 4.5 mm under dim light conditions.

75. The method of any one of claims 1-74, wherein the dosage of alpha-adrenergic antagonist is administered to the eye no more than once per day.

76. The method of any one of claims 1-75, wherein the patient experiences at least a 1 mm reduction in pupil diameter when measured under photopic conditions due to the method.

77. The method of any one of claims 1-75, wherein the patient experiences at least a 2 mm reduction in pupil diameter when measured under photopic conditions due to the method.

78. The method of any one of claims 1-75, wherein the patient experiences at least a 3 mm reduction in pupil diameter when measured under photopic conditions due to the method.

79. The method of any one of claims 1-75, wherein the patient experiences a reduction in pupil diameter ranging from about 0.5 mm to about 5 mm when measured under photopic conditions due to the method.

80. The method of any one of claims 1-79, wherein the patient experiences at least a 1 mm reduction in pupil diameter when measured under mesopic conditions due to the method.

81. The method of any one of claims 1-79, wherein the patient experiences at least a 2 mm reduction in pupil diameter when measured under mesopic conditions due to the method.

82. The method of any one of claims 1-79, wherein the patient experiences at least a 3 mm reduction in pupil diameter when measured under mesopic conditions due to the method.

83. The method of any one of claims 1-79, wherein the patient experiences a reduction in pupil diameter ranging from about 0.5 mm to about 5 mm when measured under mesopic conditions due to the method.

84. A method of treating acute angle-closure glaucoma in a patient, comprising administering to an eye of a patient in need thereof a dosage of an alpha-adrenergic antagonist selected from phentolamine or a pharmaceutically acceptable salt thereof in an amount effective to treat the acute angle-closure glaucoma.

85. The method of claim 84, wherein the patient presents with at least two conditions selected from the group consisting of severe eye pain, red eye, reduced vision, and blurred vision.

86. A method of preventing angle-closure glaucoma in a patient, comprising administering to an eye of a patient in need thereof a dosage of an alpha-adrenergic antagonist selected from phentolamine or a pharmaceutically acceptable salt thereof in an amount effective to prevent angle-closure glaucoma.

87. The method of claim 86, wherein the angle-closure glaucoma is acute angle-closure glaucoma.

88. A method of treating or preventing a narrow angle attack in a patient, comprising administering to an eye of a patient in need thereof a dosage of an alpha-adrenergic antagonist selected from phentolamine or a pharmaceutically acceptable salt thereof in an amount effective to treat or prevent the narrow angle attack.

89. The method of claim 1, wherein the method reduces the risk of a narrow angle attack in the patient.

90. The method of claim 1, wherein the method reduces the risk of angle-closure attack.

91. The method of any one of claims 84-90, further comprising administering to the eye of the patient a muscarinic acetylcholine receptor agonist.

92. The method of claim 91, wherein the muscarinic acetylcholine receptor agonist is pilocarpine or a pharmaceutically acceptable salt thereof.

93. The method of claim 91, wherein the muscarinic acetylcholine receptor agonist is a pharmaceutically acceptable salt of pilocarpine.

94. The method of claim 91, wherein the muscarinic acetylcholine receptor agonist is pilocarpine hydrochloride.

95. The method of any one of claims 91-94, wherein the muscarinic acetylcholine receptor agonist is administered to the eye of the patient at a dosage of less than about 2 mg.

96. The method of any one of claims 91-94, wherein the muscarinic acetylcholine receptor agonist is administered to the eye of the patient at a dosage of less than about 1 mg.

97. The method of any one of claims 91-94, wherein the muscarinic acetylcholine receptor agonist is administered to the eye of the patient at a dosage of less than about 0.5 mg.

98. The method of any one of claims 91-94, wherein the muscarinic acetylcholine receptor agonist is administered to the eye of the patient at a dosage of less than about 0.25 mg.

99. The method of any one of claims 91-94, wherein the muscarinic acetylcholine receptor agonist is administered to the eye of the patient at a dosage of from about 0.1 mg to about 1.5 mg.

100. The method of any one of claims 91-94, wherein the muscarinic acetylcholine receptor agonist is administered to the eye of the patient at a dosage of from about 0.1 mg to about 1.0 mg.

101. The method of any one of claims 91-94, wherein the muscarinic acetylcholine receptor agonist is administered to the eye of the patient at a dosage of from about 0.1 mg to about 0.5 mg.

102. The method of any one of claims 91-94, wherein the muscarinic acetylcholine receptor agonist is administered to the eye of the patient at a dosage of from about 0.1 mg to about 0.3 mg.

103. The method of any one of claims 91-94, wherein the muscarinic acetylcholine receptor agonist is administered to the eye of the patient at a dosage of from about 0.1 mg to about 0.2 mg.

104. The method of any one of claims 91-94, wherein the muscarinic acetylcholine receptor agonist is administered to the eye of the patient at a dosage of from about 0.1 mg to about 0.15 mg.

105. The method of any one of claims 91-94, wherein the muscarinic acetylcholine receptor agonist is administered to the eye of the patient at a dosage of from about 0.2 mg to about 0.4 mg.

106. The method of any one of claims 91-94, wherein the muscarinic acetylcholine receptor agonist is administered to the eye of the patient at a dosage of about 0.1 mg, about 0.15 mg, about 0.2 mg, about 0.25 mg, about 0.3 mg, about 0.35 mg, or about 0.4 mg.

107. The method of any one of claims 91-94, wherein the muscarinic acetylcholine receptor agonist is administered to the eye of the patient at a dosage of from about 0.3 mg to about 0.7 mg.

108. The method of any one of claims 91-107, wherein the dosage of muscarinic acetylcholine receptor agonist is administered in the form of an ophthalmic solution containing muscarinic acetylcholine receptor agonist, a lipid, and a pharmaceutically acceptable carrier.

109. The method of claim 108, wherein the muscarinic acetylcholine receptor agonist is present in the ophthalmic solution at a concentration ranging from about 0.1% (w/v) to about 4% (w/v).

110. The method of claim 108, wherein the muscarinic acetylcholine receptor agonist is present in the ophthalmic solution at a concentration ranging from about 0.1% (w/v) to about 1% (w/v).

111. The method of claim 108, wherein the muscarinic acetylcholine receptor agonist is present in the ophthalmic solution at a concentration of about 0.4% (w/v).

112. The method of claim 108, wherein the muscarinic acetylcholine receptor agonist is present in the ophthalmic solution at a concentration ranging from about 1% (w/v) to about 1.5% (w/v).

113. The method of claim 108, wherein the muscarinic acetylcholine receptor agonist is present in the ophthalmic solution at a concentration of about 1.25% (w/v).

114. The method of any one of claims 91-113, wherein the muscarinic acetylcholine receptor agonist is topically administered to the eye of the patient in the form of an eye drop.

115. The method of any one of claims 1-114, wherein the alpha-adrenergic antagonist is phentolamine or a pharmaceutically acceptable salt thereof.

116. The method of any one of claims 1-114, wherein the alpha-adrenergic antagonist is a pharmaceutically acceptable salt of phentolamine.

117. The method of any one of claims 1-114, wherein the alpha-adrenergic antagonist is phentolamine mesylate.

118. The method of any one of claims 1-114, wherein the dosage of alpha-adrenergic antagonist is in the form of an ophthalmic solution containing an aqueous pharmaceutically acceptable carrier and phentolamine or a pharmaceutically acceptable salt thereof.

119. The method of claim 118, wherein the dosage of alpha-adrenergic antagonist is in the form of an ophthalmic solution containing an aqueous pharmaceutically acceptable carrier and phentolamine mesylate.

120. The method of claim 118, wherein the dosage of alpha-adrenergic antagonist is in the form of an ophthalmic solution containing an aqueous pharmaceutically acceptable carrier and from about 0.1% (w/v) to about 2% (w/v) phentolamine mesylate.

121. The method of claim 118, wherein the dosage of alpha-adrenergic antagonist is in the form of an ophthalmic solution containing water, mannitol, and phentolamine mesylate.

122. The method of claim 118, wherein the dosage of alpha-adrenergic antagonist is in the form of an ophthalmic solution containing water, mannitol, sodium acetate, and phentolamine mesylate.

123. The method of claim 118, wherein the dosage of alpha-adrenergic antagonist is in the form of an aqueous ophthalmic solution free of a chelating agent containing:
(a) about 0.1% (w/v) to about 2% (w/v) of phentolamine mesylate;
(b) about 1% (w/v) to about 6% (w/v) of at least one polyol compound selected from the group consisting of mannitol, glycerol, and propylene glycol;
(c) about 1 mM to about 6 mM of an alkali metal acetate; and
(d) water;
wherein the solution has a pH in the range of 4 to 6 and does not contain a chelating agent.

124. The method of claim 118, wherein the dosage of alpha-adrenergic antagonist is in the form of an aqueous ophthalmic solution free of a chelating agent containing:
(a) about 0.5% (w/v) to about 2% (w/v) of phentolamine mesylate;
(b) about 1% (w/v) to about 6% (w/v) of at least one polyol compound selected from the group consisting of mannitol, glycerol, and propylene glycol;
(c) about 1 mM to about 6 mM of an alkali metal acetate; and
(d) water;
wherein the solution has a pH in the range of 4.5 to 5.5 and does not contain a chelating agent.

125. The method of claim 123 or 124, wherein the at least one polyol is mannitol.

126. The method of claim 123 or 124, wherein the solution contains 4% (w/v) mannitol.

127. The method of any one of claims 123-126, wherein the alkali metal acetate is sodium acetate.

128. The method of claim 118, wherein the dosage of alpha-adrenergic antagonist is in the form of an aqueous ophthalmic solution containing:
(a) about 0.25% (w/v) to about 2% (w/v) of phentolamine mesylate;
(b) about 3% (w/v) to about 5% (w/v) of mannitol;
(c) about 1 mM to about 6 mM of sodium acetate; and
(d) water;
wherein the solution has a pH in the range of 4.5 to 5.2 and does not contain any additional component that is a chelating agent.

129. The method of claim 118, wherein the dosage of alpha-adrenergic antagonist is in the form of an aqueous ophthalmic solution comprising: (a) about 1% (w/v) of phentolamine mesylate; (b) about 4% (w/v) mannitol; (c) about 3 mM of a buffer comprising sodium acetate; and (d) water; wherein the solution has a pH in the range of 4.5 to 5.5 and does not contain any additional component that is a chelating agent.

130. The method of any one of claims 1-118, wherein the dosage of alpha-adrenergic antagonist contains from about 0.1 mg to about 2.0 mg of phentolamine or a pharmaceutically acceptable salt thereof.

131. The method of any one of claims 1-118, wherein the dosage of alpha-adrenergic antagonist contains from about 0.5 mg to about 1.0 mg of phentolamine or a pharmaceutically acceptable salt thereof.

132. The method of any one of claims 1-129, wherein the dosage of alpha-adrenergic antagonist contains from about 0.1 mg to about 2.0 mg of phentolamine mesylate.

133. The method of any one of claims 1-129, wherein the dosage of alpha-adrenergic antagonist contains from about 0.3 mg to about 0.7 mg of phentolamine mesylate.

134. The method of any one of claims 1-129, wherein the dosage of alpha-adrenergic antagonist contains from about 0.3 mg to about 0.6 mg of phentolamine mesylate.

135. The method of any one of claims 1-129, wherein the dosage of alpha-adrenergic antagonist contains from about 0.3 mg to about 0.4 mg of phentolamine mesylate.

136. The method of any one of claims 1-129, wherein the dosage of alpha-adrenergic antagonist contains about 0.3 mg of phentolamine mesylate.

137. The method of any one of claims 1-129, wherein the dosage of alpha-adrenergic antagonist contains about 0.5 mg of phentolamine mesylate.

138. The method of any one of claims 1-129, wherein the dosage of alpha-adrenergic antagonist contains from about 0.5 mg to about 0.7 mg of phentolamine mesylate.

139. The method of any one of claims 1-129, wherein the dosage of alpha-adrenergic antagonist contains from about 0.6 mg to about 0.7 mg of phentolamine mesylate.

140. The method of any one of claims 1-129, wherein the dosage of alpha-adrenergic antagonist contains about 0.6 mg of phentolamine mesylate.

141. The method of any one of claims 1-129, wherein the dosage of alpha-adrenergic antagonist contains from about 0.8 mg to about 1.2 mg of phentolamine mesylate.

142. The method of any one of claims 1-129, wherein the dosage of alpha-adrenergic antagonist contains about 1 mg of phentolamine mesylate.

143. The method of any one of claims 1-142, wherein the alpha-adrenergic antagonist is topically administered to the eye of the patient in the form of an eye drop.

144. The method of any one of claims 1-143, wherein the patient experiences at least a 10% reduction in intraocular pressure in the eye due to the method.

145. The method of any one of claims 1-143, wherein the patient experiences at least a 20% reduction in intraocular pressure in the eye due to the method.

146. The method of any one of claims 1-143, wherein the patient experiences at least a 30% reduction in intraocular pressure in the eye due to the method.

147. The method of any one of claims 1-143, wherein the patient experiences at least a 40% reduction in intraocular pressure in the eye due to the method.

148. The method of any one of claims 1-143, wherein the patient experiences at least a 50% reduction in intraocular pressure in the eye due to the method.

149. The method of any one of claims 1-148, wherein the patient experiences at least a 5 mmHg reduction in intraocular pressure in the eye due to the method.

150. The method of any one of claims 1-148, wherein the patient experiences at least a 10 mmHg reduction in intraocular pressure in the eye due to the method.

151. The method of any one of claims 1-148, wherein the patient experiences at least a 15 mmHg reduction in intraocular pressure in the eye due to the method.

152. The method of any one of claims 1-148, wherein the patient experiences at least a 20 mmHg reduction in intraocular pressure in the eye due to the method.

153. The method of any one of claims 144-152, wherein the reduction in intraocular pressure lasts for a duration of at least 6 hours.

154. The method of any one of claims 144-152, wherein the reduction in intraocular pressure lasts for a duration of at least 12 hours.

155. The method of any one of claims 144-152, wherein the reduction in intraocular pressure lasts for a duration of at least 24 hours.

156. The method of any one of claims 1-155, wherein the patient's eye has an intraocular pressure greater than about 25 mmHg before the method is performed.

157. The method of any one of claims 1-155, wherein the patient's eye has an intraocular pressure greater than about 30 mmHg before the method is performed.

158. The method of any one of claims 1-155, wherein the patient's eye has an intraocular pressure greater than about 40 mmHg before the method is performed.

159. The method of any one of claims 1-155, wherein the patient to begin treatment is characterized as having an intraocular pressure in the range of from about 20 mmHg to about 50 mmHg.

160. The method of any one of claims 1-155, wherein the patient to begin treatment is characterized as having an intraocular pressure in the range of from about 30 mmHg to about 50 mmHg.

161. The method of any one of claims 1-155, wherein the patient to begin treatment is characterized as having an intraocular pressure in the range of from about 25 mmHg to about 30 mmHg.

162. The method of any one of claims 1-161, wherein the alpha-adrenergic antagonist and any muscarinic acetylcholine receptor agonist are administered concurrently to the eye of the patient.

163. The method of any one of claims 1-161, wherein the alpha-adrenergic antagonist and any muscarinic acetylcholine receptor agonist are administered separately to the eye of the patient.

164. The method of any one of claims 1-163, further comprising topically administering to the eye of the patient an agent that reduces eye redness.

165. The method of any one of claims 1-163, further comprising topically administering to the eye of the patient brimonidine or a pharmaceutically acceptable salt thereof.

166. The method of any one of claims 1-163, further comprising topically administering to the eye of the patient brimonidine tartrate.

167. The method of any one of claims 1-163, further comprising topically administering to the eye of the patient an ophthalmic solution comprising about 0.025% (w/w) brimonidine tartrate.

168. The method of any one of claims 1-167, wherein the patient is a human.

169. The method of any one of claims 1-168, wherein the patient's eye has a narrow angle.
